(19) United States
(12) Patent Application Publication
(10) Pub. No.: US 2021/0070862 A1
(43) Pub. Date: Mar. 11, 2021

(54) B7-H4 ANTIBODY DOSING REGIMENS

(71) Applicant: Five Prime Therapeutics, Inc., South San Francisco, CA (US)

(72) Inventors: Sandeep P. INAMDAR, South San Francisco, CA (US); Helen L. COLLINS, South San Francisco, CA (US); Xiang ZHANG, South San Francisco, CA (US); Hong XIANG, South San Francisco, CA (US)

(21) Appl. No.: 16/997,581

(22) Filed: Aug. 19, 2020

Related U.S. Application Data
(63) Continuation of application No. PCT/US2019/018963, filed on Feb. 21, 2019.
(60) Provisional application No. 62/802,100, filed on Feb. 6, 2019.
(60) Provisional application No. 62/633,527, filed on Feb. 21, 2018.

(51) Int. Cl. C07K 16/28; A61P 35/00

(52) U.S. Cl. CPC C07K 16/2827; A61K 2039/545; C07K 2317/565; A61P 35/00

(57) ABSTRACT

The present disclosure provides methods of administering antibodies and antigen-binding fragments thereof that specifically bid to human B7-H4 to a subject in need thereof, for example, a cancer patient.

### 1. FIELD

The present disclosure relates generally to methods of administering antibodies that specifically bind to human B7-H4 for the treatment of diseases such as cancer. Advantageous dose regimens are provided.

### 2. BACKGROUND

B7-H4 (also known as B7x, B7-S1, and VTCN1) is an immune regulatory molecule that shares homology with other B7 family members, include PD-L1. It is a type I transmembrane protein comprised of both IgV and IgC ectodomains. While B7-H4 expression in healthy tissues is relatively limited at the protein level, B7-H4 is expressed in several solid tumors such as gynecological carcinomas of the breast, ovary, and endometrium. Expression of B7-H4 in tumors tends to correlate with poor prognosis. The receptor for B7-H4 is unknown, but it is believed to be expressed on T cells. B7-H4 is believed to directly inhibit T cell activity.

Given the expression and function of B7-H4, antibodies that specifically bind to B7-H4 are being developed for therapies involving the modulation of B7-H4 activity, e.g., for the treatment of cancer. Accordingly, there is a need for dosing regimens for effective administration of such antibodies.

### 3. SUMMARY

Methods of administering B7-H4 antibodies and antigen-binding fragments thereof using a therapeutically effective dose regimen are provided herein.

In certain aspects, a method of treating a solid tumor in a human subject comprises administering to the subject about 0.005 to about 20 mg/kg of an antibody or antigen-binding fragment thereof that specifically binds to human B7-H4 and comprises the heavy chain variable region (VH) complementarity determining region (CDR) 1, VH CDR2, VH CDR3 and light chain variable region (VL) CDR1, VL CDR2, and VL CDR3 sequences of the 20502 antibody.

In certain aspects, a method of treating a solid tumor in a human subject comprises administering to the subject a pharmaceutical composition comprising (i) antibodies or antigen-binding fragments thereof, wherein the antibodies or antigen-binding fragments thereof specifically bind to human B7-H4 and comprise the heavy chain variable region (VH) complementarity determining region (CDR) 1, VH CDR2, VH CDR3 and light chain variable region (VL) CDR1, VL CDR2, and VL CDR3 sequences of the 20502 antibody and (ii) a pharmaceutically acceptable excipient, wherein at least 95% of the antibodies or antigen-binding fragments thereof in the composition are afucosylated, and wherein about 0.005 to about 20 mg/kg of the antibodies or antigen-binding fragments thereof are administered.

In certain aspects, the CDRs are the Kabat-defined CDRs, the Chothia-defined CDRs, or the AbM-defined CDRs. In certain aspects, the VH CDR1, VH CDR2, VH CDR3, VL CDR1, VL CDR2, and CDR3 sequences comprise the amino acid sequences set forth in SEQ ID NOs:5-10, respectively.

In certain aspects, about 20 mg/kg or 20 mg/kg of the antibody or antigen-binding fragment thereof is administered to the subject. In certain aspects, about 10 mg/kg or 10 mg/kg of the antibody or antigen-binding fragment thereof is administered to the subject. In certain aspects, about 3 mg/kg or 3 mg/kg of the antibody or antigen-binding fragment thereof is administered to the subject. In certain aspects, about 1 mg/kg or 1 mg/kg of the antibody or antigen-binding fragment thereof is administered to the subject. In certain aspects, about 0.3 mg/kg or 0.3 mg/kg of the antibody or antigen-binding fragment thereof is administered to the subject. In certain aspects, about 0.1 mg/kg or 0.1 mg/kg of the antibody or antigen-binding fragment thereof is administered to the subject. In certain aspects, wherein about 0.03 mg/kg or 0.03 mg/kg of the antibody or antigen-binding fragment thereof is administered to the subject. In certain aspects, about 0.01 mg/kg or 0.01 mg/kg of the antibody or antigen-binding fragment thereof is administered to the subject. In certain aspects, about 0.005 mg/kg or 0.005 mg/kg of the antibody or antigen-binding fragment thereof is administered to the subject.

In certain aspects, the antibody or antigen-binding fragment thereof is administered about once every three weeks.

In certain aspects, the antibody or antigen-binding fragment thereof is administered intravenously.

In certain aspects, B7-H4 has been detected in the solid tumor using immunohistochemistry (IHC) prior to the administration.

In certain aspects, the antibody or antigen-binding fragment thereof comprises a VH comprising the amino acid sequence set forth in SEQ ID NO:1 and/or a VL comprising the amino acid sequence set forth in SEQ ID NO:12. In certain aspects, the antibody or antigen-binding fragment comprises a heavy chain constant region and/or a light chain constant region. In certain aspects, the heavy chain constant region is a human immunoglobulin IgG₁ heavy chain constant region and/or the light chain constant region is a human immunoglobulin igGκ light chain constant region. In certain aspects, the antibody or antigen-binding fragment thereof comprises a heavy chain constant region comprising the amino acid sequence set forth in SEQ ID NO:25 and/or a light chain constant region comprising the amino acid sequence set forth in SEQ ID NO:23. In certain aspects, the antibody or antigen-binding fragment thereof comprises a heavy chain comprising the amino acid sequence set forth in SEQ ID NO:21 and/or a light chain comprising the amino acid sequence set forth in SEQ ID NO:22.

In certain aspects, the antibody or antigen-binding fragment thereof is a human antibody or antigen-binding fragment thereof.

In certain aspects, the antibody or antigen-binding fragment thereof is afucosylated.

In certain aspects, the antibody or antigen-binding fragment thereof is a full length antibody. In certain aspects, the antibody or antigen-binding fragment thereof is an antigen binding fragment. In certain aspects, the antigen binding fragment comprises or is a Fab, Fab′, F(ab′)₂, single chain Fv (scFv), disulfide linked Fv, V-NAR domain, IgNar, intrabody, IgGΔCH2, minibody, F(ab′)₃, tetrabody, triabody, diabody, single-domain antibody, DVD-Ig, Fcab, mAb², (scFv)₂, or scFv-Fc.

In certain aspects, fucosylation is undetectable in the composition.

In certain aspects, the solid tumor expresses B7-H4.

In certain aspects, the solid tumor is unresectable, locally advanced, or metastatic.

In certain aspects, the solid tumor is selected from the group consisting of breast cancer, ductal carcinoma, endometrial carcinoma, ovarian cancer, urothelial cancer, non-small cell lung cancer, pancreatic cancer, thyroid cancer, kidney cancer and bladder cancer. In certain aspects, the solid tumor is breast cancer, ovarian cancer, endometrial cancer, or urothelial cancer. In certain aspects, the breast cancer is advanced breast cancer. In certain aspects, the breast cancer is HER2-negative. In certain aspects, the breast cancer is triple negative breast cancer. In certain aspects, the breast cancer is hormone receptor (HR)-positive breast cancer. In certain aspects, the non-small cell lung cancer is squamous cell carcinoma. In certain embodiments, the subject has not received prior therapy with a PD-1/PD-L1 antagonist.

In certain aspects, the method further comprises monitoring the number of immune cells in the tumor. In certain aspects, the method further comprises monitoring the number of natural killer (NK) cells, CD4+ cells, and/or CD8+ cells in the tumor. In certain aspects, the method further comprises monitoring cytokine levels in the subject. In certain aspects, the method further comprises monitoring IL-2, IL-6, IL-10, TNF, and/or interferon gamma (IFNγ) levels in the subject

In certain aspects, a method of treating a solid tumor in a human subject comprises intravenously administering to the subject about once every three weeks about 20 mg/kg of an antibody thereof that specifically binds to human B7-H4 and comprises a VH comprising the amino acid sequence set forth in SEQ ID NO:11 and a VL comprising the amino acid sequence set forth in SEQ ID NO:12.

In certain aspects, a method of treating a solid tumor in a human subject comprises administering to the subject a pharmaceutical composition comprising (i) antibodies that specifically bind to human B7-H4 and comprise a VH comprising the amino acid sequence set forth in SEQ ID NO:11 and a VL comprising the amino acid sequence set forth in SEQ ID NO:12 and (ii) a pharmaceutically acceptable excipient, wherein at least 95% of the antibodies or antigen-binding fragments thereof in the composition are afucosylated, and wherein about 20 mg/kg of the antibodies or antigen-binding fragments thereof are administered intravenously about once every three weeks.

In certain aspects, the antibody comprises a heavy chain comprising the amino acid sequence set forth in SEQ ID NO:21 and a light chain comprising the amino acid sequence set forth in SEQ ID NO:22. In certain aspects, the solid tumor is breast cancer, ovarian cancer, endometrial cancer, or urothelial cancer.

### 4. BRIEF DESCRIPTION OF THE FIGURES

FIG. 1 shows the ADCC activity of fucosylated and afucosylated B7-H4 antibodies against cells with various B7-H4 expression levels. (See Example 3.)

FIG. 2 shows the effect of B7-H4 antibodies on tumor growth inhibition in mice with tumors arising from CT26 cancer cells engineered to express B7-H4. (See Example 4.)

FIG. 3 shows Phase 1a and 1b study schema. CNS=central nervous system; DLT=dose limiting toxicity; IV=intravenous; LTFU=long term follow-up; MTD=maximum tolerated dose; PD=progressive disease; Q3W=once every 3 weeks; TNBC=triple negative breast cancer; RD=recommended dose. (See Examples 7 and 8.)

### 5. DETAILED DESCRIPTION

Provided herein are methods of administering antibodies (e.g., monoclonal antibodies) and antigen-binding fragments thereof that specifically bind to B7-H4 (e.g., human B7-H4). The anti-B7-H4 antibodies and antigen-binding fragments thereof can be administered, for example, to treat a solid tumor in a subject. In a particular embodiment, about 20 mg/kg, about 10 mg/kg, about 3 mg/kg, about 1 mg/kg, about 0.3 mg/kg, about 0.1 mg/kg, about 0.03 mg/kg, about 0.01 mg/kg, or about 0.005 mg/kg of the antibody or antigen-binding fragment thereof is administered to the subject, e.g., wherein the administration occurs about every three weeks.

### 5.1 Terminology

As used herein, the term “B7-H4” refers to mammalian B7-H4 polypeptides including, but not limited to, native B7-H4 polypeptides and isoforms of B7-H4 polypeptides. “B7-H4” encompasses full-length, unprocessed B7-H4 polypeptides as well as forms of B7-H4 polypeptides that result from processing within the cell. As used herein, the term “human B7-H4” refers to a polypeptide comprising the amino acid sequence of SEQ ID NO:1. A “B7-H4 polynucleotide,” “B7-H4 nucleotide,” or “B7-H4 nucleic acid” refer to a polynucleotide encoding B7-H4.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-B7-H4 antibody,” “B7-H4 antibody” and “antibody that binds to B7-H4” refer to an antibody that is capable of specifically binding B7-H4 with sufficient affinity such that the antibody is useful as a diagnostic and/or therapeutic agent in targeting B7-H4. As used herein, the terms “specifically binding,” “immunospecifically binding,” “immunospecifically recognizing,” and “specifically recognizing” are analogous terms in the context of antibodies or antigen-binding fragments thereof. These terms indicate that the antibody or antigen-binding fragment thereof binds to an epitope via its antigen-binding domain and that the binding entails some complementarity between the antigen binding domain and the epitope. Accordingly, an antibody that “specifically binds” to human B7-H4 (SEQ ID NO:1) may also bind to B7-H4 from other species (e.g., cynomolgus monkey, mouse, and/or rat B7-H4) and/or B7-H4 proteins produced from other human alleles, but the extent of binding to an un-related, non-B7-H4 protein (e.g., other B7 protein family members such as PD-L1) is less than about 10% of the binding of the antibody to B7-H4 as measured, e.g., by a radioimmunoassay (RIA). In a specific embodiment, provided herein is an antibody or antigen-binding fragment thereof that specifically binds to human, cynomolgus monkey, mouse, and rat B7-H4.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In certain embodiments, the variable region is a human variable region. In certain embodiments, the variable region comprises rodent or murine CDRs and human framework regions (FRs). In particular embodiments, the variable region is a primate (e.g., non-human primate) variable region. In certain embodiments, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In certain aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In a specific embodiment, the CDRs of the antibodies described herein have been determined according to the Kabat numbering scheme.

Chothia refers instead to the location of the structural loops (Chothia and Lesk, J. Mol. Biol. 196.901-917 (1987)). The end of the Chothia CDR-H1 loop when numbered using the Kabat numbering convention varies between H32 and H34 depending on the length of the loop (this is because the Kabat numbering scheme places the insertions at 1135A and 1135B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” and “constant domain” are interchangeable and have their common meanings in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain. In certain aspects, an antibody or antigen-binding fragment comprises a constant region or portion thereof that is sufficient for antibody-dependent cell-mediated cytotoxicity (ADCC).

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG₁, IgG₂, IgG₃, and IgG₄. Heavy chain amino acid sequences are well known in the art. In specific embodiments, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In specific embodiments, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize antibody or antigen-binding fragment thereof specificity, affinity, and/or capability. In general, the humanized antibody or antigen-binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some embodiments, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

An “afucosylated” antibody or antigen-binding fragment thereof or an antibody or antigen-binding fragment thereof “lacking fucose” refers to an IgG1 or IgG3 isotype antibody or antigen-binding fragment thereof that lacks fucose in its constant region glycosylation. Glycosylation of human IgG1 or IgG3 occurs at Asn297 as core fucosylated biantennary complex oligosaccharide glycosylation terminated with up to 2 Gal residues. In some embodiments, an afucosylated antibody lacks fucose at Asn297. These structures are designated as G0, G1 (a 1,6 or a 1,3), or G2 glycan residues, depending on the amount of terminal Ga1 residues. See, e.g., Raju, T. S., BioProcess Int. 1: 44-53 (2003). CHO type glycosylation of antibody Fc is described, e.g., in Routier, F. F L, Glycoconjugate J. 14: 201-207 (1997).

Methods of measuring fucose include any methods known in the art. For purposes herein, fucose is detected by the method described in Example 1 of WO2015/017600, which is herein incorporated by reference in its entirety. Briefly, glycan analysis is performed by releasing glycans from the antibody (e.g., by enzymatic release), labeling the glycans with anthranilic acid (2-AA), and then purifying the labeled glycans. Normal phase HPLC with fluorescent detection is used to separate the glycans and measure the relative amount of each glycan in the antibody. The glycans may be positively identified as lacking or including fucose by mass spectrometry. In some embodiments, fucose is undetectable in a composition comprising a plurality of afucosylated antibodies or antigen-binding fragments thereof. In some embodiments, an afucosylated antibody or antigen-binding fragment thereof has enhanced affinity for Fc gamma RIIIA. In some embodiments, an afucosylated antibody or antigen-binding fragment thereof has enhanced affinity for Fc gamma RIIIA(V158). In some embodiments, an afucosylated antibody or antigen-binding fragment thereof has enhanced affinity for Fc gamma RIIIA(F158).

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1.1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{f} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In certain embodiments, the epitope to which an antibody or antigen-binding fragment thereof specifically binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody/antigen-binding fragment thereof:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

The terms “programmed cell death protein 1” and “PD-1” refer to an immunoinhibitory receptor belonging to the CD28 family. PD-1 is expressed predominantly on previously activated T-cells in vivo, and binds to two ligands, PD-L1 and PD-L2. The term “PD-1” as used herein includes human PD-1 (hPD-1), naturally occurring variants and isoforms of hPD-1, and species homologs of hPD-1. A hPD-1 sequence is

(SEQ ID NO: 30)
MQIPQAPWPVVWAVLQLGWRPGWELDSPDRPWNPPTESPALLVVTEGDNA
TFTCSFSNTSESFVLNWYRMSPSNQTDKLAAFPEDRSQPGQDCRFRVTQL
PNGRDFHMSVVRARRNDSGTYLCGAISLAPKAQIKESLRAELRVTERRAE
VPTAHPSPSPRPAGQFQTLVVGVVGGLLGSLVLLVWVLAVICSRAARGTI
GARRTGQPLKEDPSAVPVFSVDYGELDFQWREKTPEPPVPCVPEQTEYAT
IVFPSGMGTSSPARRGSADGPRSAQPLRPEDGHCSWPL.

The terms “programmed cell death 1 ligand 1” and “PD-L1” refer to one of two cell surface glycoprotein ligands for PD-1 (the other being PD-L2) that down regulate T-cell activation and cytokine secretion upon binding to PD-1. The term “PD-L” as used herein includes human PD-L1 (hPD-L1), naturally occurring variants and isoforms of hPD-1, and species homologs of hPD-L1. A hPD-L1 sequence is

(SEQ ID NO: 31)
MRIFAVFIFMTYWHLLNAFTVTVPKDLYVVEYGSNMITECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVTSE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLE
LGVALTFIFRLRKGRMMDVKKCGIQDTNSKKQSDTHLEET.

The term “PD-1/PD-L1 antagonist” refers to a moiety that disrupts the PD-1/PD-L1 signaling pathway. In some embodiments, the antagonist inhibits the PD-1/PD-L1 signaling pathway by binding to PD-1 and/or PD-L1. In some embodiments, the PD-1/PD-L1 antagonist also binds to PD-L2. In some embodiments, a PD-1/PD-L1 antagonist blocks binding of PD-1 to PD-L1 and optionally PD-L2. Nonlimiting exemplary PD-1/PD-L1 antagonists include PD-1 antagonists, such as antibodies that bind to PD-1, e.g., nivolumab (OPDIVO) and pembrolizumab (KEYTRUDA) PD-L1 antagonists, such as antibodies that bind to PD-L (e.g., atezolizumab (TECENTRIQ), durvalumab and avelumab); fusion proteins, such as AMP-224; and peptides, such as AUR-012.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention, for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in certain embodiments, the polypeptides can occur as single chains or associated chains.

As used herein, the term “host cell” can be any type of cell, e.g., a primary cell, a cell in culture, or a cell from a cell line. In specific embodiments, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that may be used to enable delivery of a drug. e.g., an anti-B7-H4 antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some embodiments, the subject is a mammal such as a non-human animal (e.g., cow, pig, horse, cat, dog, rat, mouse, monkey or other primate, etc.). In some embodiments, the subject is a cynomolgus monkey. In some embodiments, the subject is a human.

The term “therapeutically effective amount” refers to an amount of a drug, e.g., an anti-B7-H4 antibody or antigen-binding fragment thereof, effective to treat a disease or disorder in a subject. In the case of cancer, the therapeutically effective amount of the drug can reduce the number of cancer cells; reduce the tumor size or burden; inhibit, to some extent, cancer cell infiltration into peripheral organs; inhibit, to some extent, tumor metastasis; inhibit, to some extent, tumor growth; relieve, to some extent, one or more of the symptoms associated with the cancer: and/or result in a favorable response such as increased progression-free survival (PFS), disease-free survival (DFS), overall survival (OS), complete response (CR), partial response (PR), or, in some cases, stable disease (SD), a decrease in progressive disease (PD), a reduced time to progression (TTP), or any combination thereof. To the extent the drug can prevent growth and/or kill existing cancer cells, it can be cytostatic and/or cytotoxic.

Terms such as “treating,” “treatment,” “to treat,” “alleviating,” and “to alleviate” refer to therapeutic measures that cure, slow down, lessen symptoms of, and/or halt progression of a pathologic condition or disorder. Thus, those in need of treatment include those already diagnosed with or suspected of having the disorder. In certain embodiments, a subject is successfully “treated” for cancer according to the methods of the present invention if the patient shows one or more of the following: a reduction in the number of or complete absence of cancer cells; a reduction in the tumor size; inhibition of or an absence of cancer cell infiltration into peripheral organs including, for example, the spread of cancer into soft tissue and bone; inhibition or an absence of tumor metastasis; inhibition or an absence of tumor growth; relief of one or more symptoms associated with the specific cancer; reduced morbidity and mortality; improvement in quality of life; reduction in tumorigenicity, tumorigenic frequency, or tumorigenic capacity, of a tumor; reduction in the number or frequency of cancer stem cells in a tumor; differentiation of tumorigenic cells to a non-tumorigenic state: increased progression-free survival (PFS), disease-free survival (DFS), overall survival (OS), complete response (CR), partial response (PR), stable disease (SD), a decrease in progressive disease (PD), a reduced time to progression (TTP), or any combination thereof.

The terms “cancer” and “cancerous” refer to or describe the physiological condition in mammals in which a population of cells are characterized by unregulated cell growth. Examples of cancer include, but are not limited to, gynecological cancers (e.g., breast cancer (including triple negative breast cancer, ductal carcinoma, ovarian cancer, and endometrial cancer), non-small cell lung cancer, pancreatic cancer, thyroid cancer, kidney cancer (e.g., renal cell carcinoma) and bladder cancer (e.g., urothelial cell carcinoma). The cancer can be a “cancer that expresses B7-H4” or a “B7-H4 expressing cancer.” Such terms refer to a cancer comprising cells that express B7-H4. The cancer can be a solid tumor that expresses B7-H4. The cancer may be a primary tumor or may be advanced or metastatic cancer.

A “refractory” cancer is one that progresses even though an anti-tumor treatment, such as a chemotherapy, is administered to the cancer patient.

A “recurrent” cancer is one that has regrown, either at the initial site or at a distant site, after a response to initial therapy.

As used in the present disclosure and claims, the singular forms “a,” “an,” and “the” include plural forms unless the context clearly dictates otherwise.

It is understood that wherever embodiments are described herein with the language “comprising,” otherwise analogous embodiments described in terms of “consisting of” and/or “consisting essentially of” are also provided. In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited are not changed by the presence of more than that which is recited, but excludes prior art embodiments

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

### 5.2 Methods of Treating Cancer

In one aspect, presented herein are methods for treating cancer in a human subject comprising administering to a subject in need thereof an anti-B7-H4 antibody or antigen-binding fragment thereof described herein or a pharmaceutical composition thereof as described herein.

In one aspect, a method of treating cancer in a human subject comprises administering to a subject in need thereof an anti-B7-H4 antibody or antigen-binding fragment thereof described herein or a pharmaceutical composition thereof as described herein, wherein about 0.005 to about 20 mg/kg of the anti-B7-H4 antibody or antigen-binding fragment of is administered, e.g., about once every three weeks.

In one aspect, a method of treating cancer in a human subject comprises administering to a subject in need thereof an anti-B7-H4 antibody or antigen-binding fragment thereof described herein or a pharmaceutical composition thereof as described herein, wherein about 0.005 mg/kg of the anti-B7-H4 antibody or antigen-binding fragment thereof is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 0.01 mg/kg of the anti-B7-H4 antibody or antigen-binding fragment thereof is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 0.03 mg/kg of the anti-B7-H4 antibody or antigen-binding fragment thereof is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 0.1 mg/kg of the anti-B7-H4 antibody or antigen-binding fragment thereof is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 0.3 mg/kg of the anti-B7-H4 antibody or antigen-binding fragment thereof is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 1 mg/kg of the anti-B7-114 antibody or antigen-binding fragment thereof is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 3 mg/kg of the anti-B7-H4 antibody or antigen-binding fragment of is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 10 mg/kg of the anti-B7-H4 antibody or antigen-binding fragment of is administered, e.g., about once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein about 20 mg/kg of the anti-B7-H4 antibody or antigen-binding fragment thereof is administered, e.g., about once every three weeks.

In one aspect, a method of treating cancer in a human subject comprises administering to a subject in need thereof an anti-B7-H4 antibody or antigen-binding fragment thereof described herein or a pharmaceutical composition thereof as described herein, wherein 0.005 mg/kg of the anti-B7-H4 antibody or antigen-binding fragment thereof is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 0.01 mg/kg of the anti-B7-H4 antibody or antigen-binding fragment thereof is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 0.03 mg/kg of the anti-B7-H4 antibody or antigen-binding fragment thereof is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 0.1 mg/kg of the anti-B7-H4 antibody or antigen-binding fragment thereof is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 0.3 mg/kg of the anti-B7-H4 antibody or antigen-binding fragment thereof is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 1 mg/kg of the anti-B7-H4 antibody or antigen-binding fragment thereof is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 3 mg/kg of the anti-B7-H4 antibody or antigen-binding fragment of is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 10 mg/kg of the anti-B7-H4 antibody or antigen-binding fragment of is administered, e.g., once every three weeks. In one aspect, a method of treating cancer in a human subject comprises administering to a subject in need thereof an anti-B7-H4 antibody or antigen-binding fragment thereof described herein or a pharmaceutical composition thereof as described herein, wherein 20 mg/kg of the anti-B7-H4 antibody or antigen-binding fragment thereof is administered, e.g., once every three weeks.

According to the methods provided herein, the anti-B7-H4 antibody or antigen binding fragment thereof, or the pharmaceutical composition comprising anti-B7-H4 antibodies or antigen-binding fragments thereof, can be administered intravenously.

In a certain embodiment, provided herein are methods of treating a cancer selected from the group consisting of: breast cancer (e.g., advanced breast cancer, triple negative breast cancer, or ductal carcinoma), endometrial carcinoma, ovarian cancer, urothelial cancer, non-small cell lung cancer (e.g., squamous cell carcinoma), pancreatic cancer, thyroid cancer, kidney cancer (e.g., renal cell carcinoma), and bladder cancer (e.g., urothelial cell carcinoma) In a certain embodiment, provided herein are methods of treating advanced breast cancer (including triple-negative breast cancer), ovarian cancer, endometrial cancer, or urothelial cancer. In a certain embodiment, provided herein are methods of treating a breast cancer. In a certain embodiment, provided herein are methods of treating a hormone-receptor (HR)-positive breast cancer. In a certain embodiment, provided herein are methods of treating an ovarian cancer. In a certain embodiment, provided herein are methods of treating an endometrial cancer. In a certain embodiment, provided herein are methods of treating a urothelial cancer. In a certain embodiment, provided herein, the subject has not received prior therapy with a PD-1/PD-L1 antagonist. In certain embodiments, such methods comprise administering an anti-B7-H4 antibody or antigen-binding fragment thereof provided herein, or a pharmaceutical composition comprising anti-B7-H4 antibodies or antigen-binding fragments thereof provided herein, to a patient (e.g., a human patient) in need thereof.

In some embodiments, the cancer is a B7-H4 expressing cancer. In certain embodiments, the cancer is a solid tumor solid tumor that expresses B7-H4. In certain embodiments, B7-H4 has been detected (e.g., using immunohistochemistry (IHC)) in a biological sample obtained from the subject.

A biological sample may be any biological sample obtained from a subject, cell line, tissue, or other source of cells potentially expressing B7-H4. Methods for obtaining tissue biopsies and body fluids from humans are well known in the art. Biological samples include peripheral mononuclear blood cells. A biological sample may also be a blood sample, in which circulating tumor cells (or “CTCs”) may express B7-H4 and be detected.

Assaying for the expression level of B7-H4 protein is intended to include qualitatively or quantitatively measuring or estimating the level of a B7-H4 protein in a first biological sample either directly (e.g., by determining or estimating absolute protein level) or relatively (e.g., by comparing to the protein level in a second biological sample). B7-H4 polypeptide expression level in the first biological sample can be measured or estimated and compared to a standard B7-H4 protein level, the standard being determined from a second biological sample that is not diseased or being determined by averaging levels from a population of samples that are not diseased. As will be appreciated in the art, once the “standard” B7-H4 polypeptide level is known, it can be used repeatedly as a standard for comparison.

In another embodiment, an anti-B7-H4 antibody or antigen-binding fragment thereof, or pharmaceutical composition, is administered to a patient (e.g., a human patient) diagnosed with cancer to increase the proliferation of T cells, CD4⁺ T cells, or CD8⁺ T cells in the patient. In another embodiment, an anti-B7-H4 antibody or antigen-binding fragment thereof, or pharmaceutical composition, is administered to a patient (e.g., a human patient) diagnosed with cancer to increase interferon-gamma (IFNγ) production in the patient. In another embodiment, an anti-B7-H4 antibody or antigen-binding fragment thereof, or pharmaceutical composition, is administered to a patient (e.g., a human patient) diagnosed with cancer to block the inhibitory activity of B7-H4 against T cells in the patient. In another embodiment, an anti-B7-H4 antibody or antigen-binding fragment thereof, or pharmaceutical composition, is administered to a patient (e.g., a human patient) diagnosed with cancer to deplete B7-H4 expressing cancer cells in the patient.

In some embodiments, the present invention relates to an anti-B7-H4 antibody or antigen-binding fragment thereof or pharmaceutical composition provided herein for use as a medicament, wherein the medicament is for administration at about 0.005 mg/kg to about 20 mg/kg (e.g., about 0.005 mg/kg, about 0.01 mg/kg, about 0.03 mg/kg, about 0.1 mg/kg, about 0.3 mg/kg, about 1 mg/kg, about 3 mg/kg, about 10 mg/kg, or about 20 mg/kg) of the antibody or antigen-binding fragment thereof. In some aspects, the present invention relates to an antibody or antigen-binding fragment thereof or pharmaceutical composition provided herein, for use in a method for the treatment of cancer wherein about 0.005 mg/kg to about 20 mg/kg (e.g., about 0.005 mg/kg, about 0.01 mg/kg, about 0.03 mg/kg, about 0.1 mg/kg, about 0.3 mg/kg, about 1 mg/kg, about 3 mg/kg, about 10 mg/kg, or about 20 mg/kg) of the antibody or antigen-binding fragment thereof is administered. In some aspects, the present invention relates to an antibody or antigen-binding fragment thereof or pharmaceutical composition provided herein, for use in a method for the treatment of cancer in a subject, comprising administering to the subject about 0.005 mg/kg to about 20 mg/kg (e.g., about 0.005 mg/kg, about 0.01 mg/kg, about 0.03 mg/kg, about 0.1 mg/kg, about 0.3 mg/kg, about 1 mg/kg, about 3 mg/kg, about 10 mg/kg, or about 20 mg/kg) of an antibody or antigen-binding fragment thereof or pharmaceutical composition provided herein.

In some embodiments, the present invention relates to an anti-B7-H4 antibody or antigen-binding fragment thereof or pharmaceutical composition provided herein for use as a medicament, wherein the medicament is for administration at 0.005 mg/kg to 20 mg/kg (e.g., 0.005 mg/kg, 0.01 mg/kg, 0.03 mg/kg, 0.1 mg/kg, 0.3 mg/kg, 1 mg/kg, 3 mg/kg, 10 mg/kg, or 20 mg/kg) of the antibody or antigen-binding fragment thereof. In some aspects, the present invention relates to an antibody or antigen-binding fragment thereof or pharmaceutical composition provided herein, for use in a method for the treatment of cancer wherein 0.005 mg/kg to 20 mg/kg (e.g., 0.005 mg/kg, 0.01 mg/kg, 0.03 mg/kg, 0.1 mg/kg, 0.3 mg/kg, 1 mg/kg, 3 mg/kg, 10 mg/kg, or 20 mg/kg) of the antibody or antigen-binding fragment thereof is administered. In some aspects, the present invention relates to an antibody or antigen-binding fragment thereof or pharmaceutical composition provided herein, for use in a method for the treatment of cancer in a subject, comprising administering to the subject 0.005 mg/kg to 20 mg/kg (e.g., 0.005 mg/kg, 0.01 mg/kg, 0.03 mg/kg. 0.1 mg/kg, 0.3 mg/kg, 1 mg/kg. 3 mg/kg, 10 mg/kg, or 20 mg/kg) of an antibody or antigen-binding fragment thereof or pharmaceutical composition provided herein.

### 5.3 B7-H4 Antibodies and Antigen-Binding Fragments Thereof

Provided herein are methods of treating cancer in a human subject comprising administering to the subject antibodies (e.g., monoclonal antibodies, such as chimeric, humanized, or human antibodies) and antigen-binding fragments thereof which specifically bind to B7-H4 (e.g., human B7-H4). Exemplary B7-H4 antibodies and antigen-binding fragments thereof that can be used in the methods provided herein are known in the art. The amino acid sequences for human, cynomolgus monkey, murine, and rat B7-H4 are known in the art and also provided herein as represented by SEQ ID NOs:1-4, respectively.

Human B7-H4:
(SEQ ID NO: 1)
MASLGQILFWSIISIIIILAGAIALIIGEGISGRHSITVTTVASAGNIGE
DGILSCTFEPDIKLSDIVIQWLKEGVLGLVHEFKEGKDELSEQDEMFRGR
TAVFADQVIVGNASLRLKNVQLTDAGTYKCYIITSKGKGNANLEYKTGAF
SMPEVNVDYNASSETLRCEAPRWFPQPTVVWASQVDQGANFSEVSNTSFE
LNSENVTMKVVSVLYNVTINNTYSCMIENDIAKATGDIKVTESEIKRRSH
LQLLNSKASLCVSSFFAISWALLPLSPYLMLK
Cynomolgus monkey B7-114:
(SEQ ID NO: 2)
MASLGQILFWSIISIIFILAGAIALIIGFGISGRHSITVTTVASAGNIGE
DGILSCTFEPDIKLSDIVIQWLKEGVIGLVHEFKEGKDELSEQDEMFRGR
TAVFADQVIVGNASLRLKNVQLTDAGTYKCYIITSKGKGNANLEYKTGAF
SMPEVNVDYNASSETLRCEAPRWFPQRTVVWASQVDQGANFSEVSNTSFE
LNSENVTMKVVSVLYNVTINNTYSCMIENDIAKATGDIKVTESEIKRRSH
LQLLNSKASLCVSSFLAISWALLPLAPYLMLK
Murine B7-H4
(SEQ ID NO: 3)
MASLGQIIFWSIINIIIILAGAIALIIGFGISGKHFITVTTFTSAGNIGE
DGTLSCTFEPDIKLNGIVIQWLKEGIKGLVHEFKEGKDDLSQQHEMFRGR
TAVFADQVVVONASLRLKNVQLTDAGTYTCYIRTSKGKGNANLEYKTGAF
SMPEINVDYNASSESLRCEAPRWFPQPTVAWASQVDQGANFSEVSNTSFE
LNSENVTMKVVSVLYNVTINNTYSCMIENDIAKATGDIKVTDSEVKRRSQ
LQLLNSGPSPCVFSSAFVAGWALLSLSCCLMLR
Rat B7-114
(SEQ ID NO: 4)
MASLGQIIFWSIINVIIILAGAIVLIIGFGISGKHFITVTTFTSAGNIGE
DGTLSCTFEPDIKLNGIVIQWLKEGIKGLVHEFKEGKDDLSQQHEMFRGR
TAVFADQVVVGNASLRLKNVQLTDAGTYTCYIHTSKGKGNANLEYKTGAF
SMPEINVDYNASSESLRCEAPRWFPQPTVAWASQVDQGANFSEVSNTSFE
LNSENVTMKVVSVLYNVTINNTYSCMIENDIAKATGDIKVTDSEVKRRSQ
LELLNSGPSPCVSSVSAAGWALLSLSCCLMLR

In certain embodiments, an antibody or antigen-binding fragment thereof for use in the methods described herein specifically binds to human B7-H4. In certain embodiments, an antibody or antigen-binding fragment thereof for use in the methods described herein specifically binds to human and cynomolgus monkey B7-H4. In certain embodiments, an antibody or antigen-binding fragment thereof for use in the methods described herein specifically binds to human, murine, and rat B7-H4. In certain embodiments, an antibody or antigen-binding fragment thereof for use in the methods described herein specifically binds to human, cynomolgus monkey, murine, and rat B7-H4.

B7-H4 contains an IgC ectodomain (amino acids 153-241 of SEQ ID NO:1) and an IgV ectodomain (amino acids 35-146 of SEQ ID NO:1). In certain embodiments, an antibody or antigen-binding fragment thereof for use in the methods described herein specifically binds to the IgV domain of human B7-H4. Accordingly, provided herein are methods of administering antibodies and antigen-binding fragments thereof that specifically bind to a polypeptide consisting of amino acids 35-146 of SEQ ID NO:1.

In certain embodiments, an antibody or antigen-binding fragment thereof for use in the methods described herein specifically binds to human B7-H4 and comprises the six CDRs of the 20502 antibody listed as provided in Tables 1 and 2. “20502” refers to the 20502 antibody, described herein.

TABLE 1
VH CDR Amino Acid Sequences ¹

| Anti- | VH CDR1 | VH CDR2 | VH CDR3 |
|---|---|---|---|
| body | (SEQ ID NO:) | (SEQ ID NO:) | (SEQ ID NO:) |
| 20502 | GSIKSGSYYWG | MYYSGSTYYNPSLRS | AREGSYPNQFDP |
| | (SEQ ID NO: 5) | (SEQ ID NO: 6) | (SEQ ID NO: 7) |

¹ The VH CDRs in Table 1 are determined according to Kabat.

TABLE 2
VL CDR Amino Acid Sequences ²

| Anti- | VL CDR1 | VL CDR2 | VL CDR3 |
|---|---|---|---|
| body | (SEQ ID NO:) | (SEQ ID NO:) | (SEQ ID NO:) |
| 20502 | RASQSVSSNLA | GASTRAT | QQYITISFPFT |
| | (SEQ ID NO: 8) | (SEQ ID NO: 9) | (SEQ ID NO: 10) |

² The VL CDRs in Table 2 are determined according to Kabat.

In certain embodiments, an antibody or antigen-binding fragment thereof for use in the methods described herein specifically binds to human B7-H4 and comprises the VH of the 20502 antibody listed in Table 3.

TABLE 3
Variable Heavy Chain (VH) Amino Acid Sequences

| Antibody | VH Amino Acid Sequence (SEQ ID NO) |
|---|---|
| 20502 | QLQLQESGPGLVKPSETLSLTCTVSGGSIKSGSYYWGWI |
| | RQPPGKGLEWIGNIYYSGSTYYNPSLRSRVTISVDTSKN |
| | QFSLKLSSVTAADTAVYYCAREGSYPNQFDPWGQGTLVT |
| | VSS (SEQ ID NO: 11) |

In certain embodiments, an antibody or antigen-binding fragment thereof for use in the methods described herein specifically binds to human B7-H4 and comprises the VL of the 20502 listed in Table 4.

TABLE 4
Variable Light Chain (VL) Amino Acid Sequences

| Antibody | VL Amino Acid Sequence (SEQ ID NO) |
|---|---|
| 20502 | EIVMTQSPATLSVSPGERATLSCRASQSVSSNLAWYQQK |
| | PGQAPRLLIYGASTRATGIPARFSGSGSGTEFTLTISSL |
| | QSEDFAVYYCQQYHSFPFTFGGGTKVEIK |
| | (SEQ ID NO: 12) |

In certain embodiments, an antibody or antigen-binding fragment thereof for use in the methods described herein specifically binds to human B7-H4 and comprises the VH and the VL of the 20502 antibody listed in Tables 3 and 4.

In certain embodiments, an antibody or antigen-binding fragment thereof for use in the methods described herein specifically binds to human B7-H4 and comprises the VH framework regions of the 20502 antibody listed in Table 5.

TABLE 5
VH FR Amino Acid Sequences ³

| | | VH FR2 | VH FR3 | VH FR4 |
|---|---|---|---|---|
| Anti- | VH FR1 | (SEQ | (SEQ | (SEQ |
| body | (SEQ ID NO:) | ID NO:) | ID NO:) | ID NO:) |
| 20502 | QLQLQESGPGLVK | WIRQPPGKG | RVTISVDT | WGQGTL |
| | PSETLSLTCTVSG | LEWIG | SKNQFSLK | VTVSS |
| | (SEQ ID NO: 13) | (SEQ ID | LSSVTAAD | (SEQ ID |
| | | NO: 14) | TAVYYC | NO: 16) |
| | | | (SEQ ID | |
| | | | NO: 15) | |

³ The VH framework regions deszribed in Table 5 are determined based upon the boundaries of the Kabat numbering system for CDRs.
Accordingly, the VH CDRs are determined by Kabat and the framework regions are the amino acid residues surrounding the CDRs in the variable region in the format FR1, CDR1, FR2, CDR2, FR3, CDR3, and FR4.

In certain embodiments, an antibody or antigen-binding fragment thereof for use in the methods described herein specifically binds to human B7-H4 and comprises the VL framework regions of the 20502 antibody listed in Table 6.

TABLE 6
VL FR Amino Acid Sequences ⁴

| | VL FR1 | VL FR2 | | VL FR4 |
|---|---|---|---|---|
| Anti- | (SEQ | (SEQ | VL FR3 | (SEQ |
| body | ID NO:) | ID NO:) | (SEQ ID NO:) | ID NO:) |
| 20502 | EIVIVITQSP | WYQQKPGQ | GIPARFSGSGSGT | FGGGIRV |
| | ATTSVSPGER | APRLLIY | IEFTLTISSLOSE | EIK |
| | ATLSC | | | |
| | (SEQ ID | (SEQ ID | DFAVYYC (SEQ | (SEQ ID |
| | NO: 17) | NO: 18) | ID NO: 20) | NO: 19) |

⁴ The VL framework regions described in Table 6 are determined based upon the boundaries of the Kabat numbering system for CDRs.
Accordingly, the VL CDRs are determined by Kabat and the framework regions are the amino acid residues surrounding the CDRs in the variable region in the format FR1, CDR1, FR2, CDR2, FR3, CDR3, and F14.

In certain embodiments, an antibody or antigen-binding fragment thereof for use in the methods described herein specifically binds to human B7-H4 and comprises the four VH framework regions and the four VL framework regions of the 20502 antibody listed in Tables 5 and 6.

In certain embodiments, an antibody or antigen-binding fragment thereof for use in the methods described herein specifically binds to human B7-H4 and comprises the heavy chain sequence of the 20502 antibody listed in Table 7.

TABLE 7
Full-length heavy chain amino acid sequences

| | Full-Length Heavy Chain Amino |
|---|---|
| Antibody | Acid Sequence (SEQ ID NO) |
| 20502 | QLQLQESGPGLVKPSETLSLTCTVSGGSIKSGSYYWGWI |
| | RQPPGKGLEWIGNIYYSGSTYYNPSLRSRVTISVDTSKN |
| | QFSLKLSSVTAADTAVYYCAREGSYPNQFDPWGQGTLVT |
| | VSSASTKGPSVFPLAPSSKSTSGGTAALGCLVKDYFPEP |
| | VTVSWNSGALTSGVHTFPAVLQSSGLYSLSSVVTVPSSS |
| | LGTQTYICNVNHKPSNTKVDKKVEPKSCDKTHTCPPCPA |
| | PELLGGPSVFLFPPKPKLYTLMISRTPEVTCVVVDVSHE |
| | DPEVKFNWYVDGVEVHNAKTKPREEQYNSTYRVVSVLTV |
| | LHQDWLNGKEYKCKVSNKALPAPIEKTISKAKGQPREPQ |
| | VYTLPPSRDELTKNQVSLTCLVKGFYPSDIAVEWESNGQ |
| | PENNYKTTPPVLDSDGSFFLYSKLTVDKSRWQQGNVFSC |
| | SVMHEALHNHYTQKSLSLSPGK (SEQ ID NO: 21) |

In certain embodiments, an antibody or antigen-binding fragment thereof for use in the methods described herein specifically binds to human B7-H4 and comprises the light chain sequence of the 20502 antibody listed in Table 8.

TABLE 8
Full-length light chain amino acid sequences

| | Full-Length Light Chain Amino |
|---|---|
| Antibody | Acid Sequence (SEQ ID NO) |
| 20502 | EIVMTQSPATLSVSPGERATLSCRASQSVSSNLAWYQQKP |
| | GQAPRLLIYGASTRATGIPARFSGSGSGTEFTLTISSLQS |
| | EDFAVYYCQQYHSFPFTEGGGTKVEIKRTVAAPSVFIFPP |
| | SDEQLKSGTASVVCLLNNFYPREAKVQWKVDNALQSGNSQ |
| | ESVTEQDSKDSTYSLSSTLTLSKADYEKHKVYACENTTHQ |
| | GLSSPVTKSFNRGEC (SEQ ID NO: 22) |

In certain embodiments, an antibody or antigen-binding fragment for use in the methods described herein specifically binds to human B7-H4 and comprises the heavy chain sequence and the light chain sequence of the 20502 antibody listed in Tables 7 and 8.

In certain aspects, an antibody or antigen-binding fragment thereof for use in the methods described herein is described by its VL domain alone, or its VH domain alone, or by its 3 VL CDRs alone, or its 3 VH CDRs alone. See, for example, Rader C et al., (1998) PNAS 95: 8910-8915, which is incorporated herein by reference in its entirety, describing the humanization of the mouse anti-αvβ antibody by identifying a complementing light chain or heavy chain, respectively, from a human light chain or heavy chain library, resulting in humanized antibody variants having affinities as high or higher than the affinity of the original antibody. See also Clackson T et al., (1991) Nature 352: 624-628, which is incorporated herein by reference in its entirety, describing methods of producing antibodies that specifically bind a specific antigen by using a specific VL domain (or VH domain) and screening a library for the complementary VH domain or (VL domain). The screen produced 14 new partners for a specific VH domain and 13 new partners for a specific VL domain, which were strong binders, as determined by ELISA. See also Kim S J & Hong H J, (2007) J Microbiol 45: 572-577, which is incorporated herein by reference in its entirety, describing methods of producing antibodies that specifically bind a specific antigen by using a specific VH domain and screening a library (e.g., human VL library) for complementary VL domains; the selected VL domains in turn could be used to guide selection of additional complementary (e.g., human) VH domains.

In certain aspects, the CDRs of an antibody or antigen-binding fragment thereof can be determined according to the Chothia numbering scheme, which refers to the location of immunoglobulin structural loops (see, e.g., Chothia C & Lesk A M, (1987), J Mol Biol 196: 901-917; Al-Lazikani B e al., (1997) J Mol Biol 273.927-948; Chothia C et al., (1992) J Mol Biol 227: 799-817; Tramontano A et al., (1990) J Mol Biol 215(1): 175-82; and U.S. Pat. No. 7,709,226). Typically, when using the Kabat numbering convention, the Chothia CDR-H1 loop is present at heavy chain amino acids 26 to 32, 33, or 34, the Chothia CDR-H2 loop is present at heavy chain amino acids 52 to 56, and the Chothia CDR-H3 loop is present at heavy chain amino acids 95 to 102, while the Chothia CDR-L1 loop is present at light chain amino acids 24 to 34, the Chothia CDR-L2 loop is present at light chain amino acids 50 to 56, and the Chothia CDR-L3 loop is present at light chain amino acids 89 to 97. The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34).

In certain aspects, provided herein are methods of administering antibodies and antigen-binding fragments thereof that specifically bind to B7-H4 (e.g., human B7-H4) and comprise the Chothia VII and VL CDRs of the 20502 antibody listed in Tables 3 and 4. In certain embodiments, provided herein are methods of administering antibodies or antigen-binding fragments thereof that specifically bind to B7-H4 (e.g., human B7-H4) and comprise one or more CDRs, in which the Chothia and Kabat CDRs have the same amino acid sequence. In certain embodiments, provided herein are methods of administering antibodies and antigen-binding fragments thereof that specifically bind to B7-H4 (e.g., human B7-H4) and comprise combinations of Kabat CDRs and Chothia CDRs.

In certain aspects, the CDRs of an antibody or antigen-binding fragment thereof can be determined according to the IMGT numbering system as described in Lefranc M-P, (1999) The Immunologist 7: 132-136 and Lefranc M-P et al., (1999) Nucleic Acids Res 27: 209-212. According to the IMGT numbering scheme, VH-CDR1 is at positions 26 to 35, VH-CDR2 is at positions 51 to 57, VH-CDR3 is at positions 93 to 102, VL-CDR1 is at positions 27 to 32, VL-CDR2 is at positions 50 to 52, and VL-CDR3 is at positions 89 to 97. In a particular embodiment, provided herein are methods of administering antibodies and antigen-binding fragments thereof that specifically bind to B7-H4 (e.g., human B7-H4) and comprise the IMGT VH and VL CDRs of the 20502 antibody listed in Tables 3 and 4, for example, as described in Lefranc M-P (1999) supra and Lefranc M-P et al., (1999) supra).

In certain aspects, the CDRs of an antibody or antigen-binding fragment thereof can be determined according to MacCallum R M et al., (1996) J Mol Biol 262: 732-745. See also, e.g., Martin A. “Protein Sequence and Structure Analysis of Antibody Variable Domains,” in Antibody Engineering, Kontermann and Dübel, eds., Chapter 31, pp. 422-439, Springer-Verlag, Berlin (2001). In a particular embodiment, provided herein are methods of administering antibodies or antigen-binding fragments thereof that specifically bind to B7-H4 (e.g., human B7-H4) and comprise VH and VL CDRs of the 20502 antibody listed in Tables 3 and 4 as determined by the method in MacCallum R M et al.

In certain aspects, the CDRs of an antibody or antigen-binding fragment thereof can be determined according to the AbM numbering scheme, which refers AbM hypervariable regions which represent a compromise between the Kabat CDRs and Chothia structural loops, and are used by Oxford Molecular's AbM antibody modeling software (Oxford Molecular Group, Inc.). In a particular embodiment, provided herein are methods of administering antibodies or antigen-binding fragments thereof that specifically bind to B7-H4 (e.g., human B7-H4) and comprise VH and VL CDRs of the 20502 antibody listed in Tables 3 and 4 as determined by the AbM numbering scheme.

In specific aspects, provided herein are methods of administering antibodies that comprise a heavy chain and a light chain.

With respect to the light chain, in a specific embodiment, the light chain of an antibody described herein is a kappa light chain. The constant region of a human kappa light chain can comprise the following amino acid sequence:

(SEQ ID NO: 23)
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

The constant region of a human kappa light chain can be encoded by the following nucleotide sequence:

(SEQ ID NO: 24)
CGGACCGTGGCTGCACCATCTGTCTTCATCTTCCCGCCATCTGATGAGCA
GTTGAAATCTGGAACTGCCTCTGTTGTGTGCCTGCTGAATAACTTCTATC
CCAGAGAGGCCAAAGTACAGTGGAAGGTGGATAACGCCCTCCAATCGGGT
AACTCCCAGGAGAGTGTCACAGAGCAGGACAGCAAGGACAGCACCTACAG
CCTCAGCAGCACCCTGACGCTGAGCAAAGCAGACTAcGAGAAACACAAAG
TCTACGCCTGCGAAGTCACCCATCAGGGCCTGAGCTCGCCCGTCACAAAG
AGCTTCAACAGGGGAGAGTGT.

In a particular embodiment, an antibody which immunospecifically binds to a B7-H4 polypeptide (e.g., human B7-H4) for use in the methods described herein comprises a light chain wherein the amino acid sequence of the VL domain comprises the sequence set forth in Table 4, and wherein the constant region of the light chain comprises the amino acid sequence of a human kappa light chain constant region.

In a particular embodiment, an antibody which immunospecifically binds to B7-H4 (e.g., human B7-H4) for use in the methods described herein comprises a heavy chain wherein the amino acid sequence of the VH domain comprises the amino acid sequence set forth in Table 3 and wherein the constant region of the heavy chain comprises the amino acid sequence of a human gamma (γ) heavy chain constant region.

The constant region of a human IgG₁ heavy chain can comprise the following amino acid sequence:

(SEQ ID NO: 25)
ASTKGPSVFPLAPSSKSTSGGTAALGCLNKDYFPEPVTVSWNSGALTSGV
HTFPAVLQSSGLYSLSSVVTVPSSSLGTQTYICNVNHKPSNTKVTDKKVE
PKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNG
KEYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK.

The constant region of a human IgG₁ heavy chain can be encoded by the following nucleotide sequence:

(SEQ ID NO: 26)
GCCTCCACCAAGGGCCCATCGGTCTTCCCCCTGGCACCCTCCTCCAAGAG
CACCTCTGGGGGCACAGCGGCCCTGGGCTGCCTGGTCAAGGACTACTTCC
CCGAACCGGTGACGGTGTCGTGGAACTCAGGCGCCCTGACCAGCGGCGTG
CACACCTTCCCGGCTGTCCTACAGTCCTCAGGACTCTACTCCCTCAGCAG
CGTGGFGACCGTGCCCTCCAGCAGCTTGGGCACCCAGACCTCATCTGCAA
CGTGAATCACAAGCCCAGCAACACCAAGGTGGACAAGAAAGTTGAGCCCA
AATCTTGTGACAAAACTCACACATGCCCACCGTGCCCAGCACCTGAACTC
CTGGGGGGACCGTCAGTCTTCCTCTTCCCCCCAAAACCCAAGGACACCCT
CATGATCTCCCGGACCCCTGAGGTCACATGCGTGGTGGTGGACGTGAGCC
ACGAAGACCCTGAGGTCAAGTTCAACTGGTACGTGGACGGCGTGGAGGTG
CATAATGCCAAGACAAAGCCGCGGGAGGAGCAGTACAACAGCACGTACCG
GGTGGTCAGCGTCCTCACCGTCCTGCACCAGGACTGGCTGAATGGCAAGG
AGTACAAGTGCAAGGTCTCCAACAAAGCCCTCCCAGCCCCCATCGAGAAA
ACCATCTCCAAAGCCAAAGGGCAGCCCCGAGAACCACAGGTGTACACCCT
GCCCCCATCCCGGGATGAGCTGACCAAGAACCAGGTCAGCCTGACCTGCC
TGGTCAAAGGCTTCTATCCCAGCGACATCGCCGTGGAGTGGGAGAGCAAT
GGGCAGCCGGAGAACAACTACAAGACCACGCCTCCCGTGCTGGACTCCGA
CGGCTCCTTCTTCCTCTACAGCAAGCTCACCGTGGACAAGAGCAGGTGGC
AGCAGGGGAACGTCTTCTCATGCTCCGTGATGCATGAGGCTCTGCACAAC
CACTACACGCAGAAGAGCCTCTCCCTGTCTCCGGGTAAA.

In a specific embodiment, an antibody which immunospecifically binds to B7-H4 (e.g., human B7-H4) for use in the methods described herein comprises a VH domain and a VL domain comprising an amino acid sequence of any VH and VL domain described herein, and wherein the constant regions comprise the amino acid sequences of the constant regions of an IgG (e.g., a human IgG) immunoglobulin molecule. In another specific embodiment, an antibody which immunospecifically binds to B7-H4 (e.g., human B7-H4) for use in the methods described herein comprises a VH domain and a VL domain comprising an amino acid sequence of any VH and VL domain described herein, and wherein the constant regions comprise the amino acid sequences of the constant regions of an IgG₁ (e.g. human IgG₁ immunoglobulin molecule.

Antibodies with reduced fucose content have been reported to have an increased affinity for Fc receptors, such as, e.g., FcγRIIIA. Accordingly, in certain embodiments, an antibody or antigen-binding fragment thereof for use in the methods described herein has reduced fucose content or lacks fucose (i.e., is “afucosylated”). Such antibodies or antigen-binding fragments thereof can be produced using techniques known to one skilled in the art. For example, they can be expressed in cells deficient or lacking the ability to fucosylate. In a specific example, cell lines with a knockout of both alleles of the α1,6-fucosyltransferase gene (FUT8) can be used to produce antibodies or antigen-binding fragments thereof with reduced fucose content. The Potelligent® system (Lonza) is an example of such a system that can be used to produce antibodies and antigen-binding fragments thereof with reduced fucose content. Alternatively, antibodies or antigen-binding fragments thereof with reduced fucose content or no fucose content can be produced by, e.g.: (i) culturing cells under conditions which prevent or reduce fucosylation; (ii) posttranslational removal of fucose (e.g., with a fucosidase enzyme); (iii) post-translational addition of the desired carbohydrate, e.g., after recombinant expression of a non-glycosylated glycoprotein; or (iv) purification of the glycoprotein so as to select for antibodies or antigen-binding fragments thereof which are not fucosylated. See, e.g., Longmore G D & Schachter H (1982) Carbohydr Res 100: 365-92 and Imai-Nishiya H et al., (2007) BMC Biotechnol. 7: 84 for methods for producing antibodies thereof with no fucose content or reduced fucose content.

In some embodiments, an afucosylated B7-H4 antibody or antigen-binding fragment thereof has enhanced ADCC activity in vitro compared to fucosylated B7-14 antibodies or antigen-binding fragments thereof having the same amino acid sequence. In some embodiments, the afucosylated B7-H4 antibodies or antigen-binding fragments thereof cause specific lysis that is at least 10, at least 15, at least 20, at least 25, at least 30, at least 35, at least 40, at least 45, at least 50, at least 60, at least 65, at least 70, or at least 75 percentage points greater than specific lysis with fucosylated B7-H4 antibodies. Specific lysis may be determined as described in Example 2 herein.

In some embodiments, the B7-H4 antibody or antigen-binding fragment thereof has enhanced affinity for Fc gamma RIIIA compared to fucosylated B7-H4 antibodies or antigen-binding fragments thereof having the same amino acid sequence. In some embodiments, the afucosylated B7-H4 antibodies or antigen-binding fragments thereof bind to Fc gamma RIIIA with at least 2-fold, at least 3-fold, at least 4-fold, at least 5-fold, at least 7-fold, at least 10-fold, at least 12-fold, at least 15-fold, at least 17-fold, or at least 20-fold greater affinity than fucosylated B7-H4 antibodies or antigen-binding fragments thereof. In some embodiments, affinity for Fc gamma RIIIA is determined using surface plasmon resonance. In some embodiments, Fc gamma RIIIA is selected from Fc gamma RIIIA(V158) and Fc gamma RIIIA(F158). In some embodiments, Fc gamma RIIIA is Fc gamma RIIIA(V158).

In some embodiments, the presence of fucose can be determined by a method comprising high performance liquid chromatography (HPLC), capillary electrophoresis, or MALDI-TOF mass spectrometry.

In specific embodiments, an antibody or antigen-binding fragment thereof (i) comprises the CDR sequences of 20502, the VH and VL sequences of 20502, or the heavy and light chain sequences of 20502 and (ii) is afucosylated.

In specific embodiments, a composition comprises antibodies or antigen-binding fragments thereof that (i) comprises the CDR sequences of 20502, the VH and VL sequences of 20502, or the heavy and light chain sequences of 20502 and (ii) are afucosylated, e.g., wherein at least 95% of the antibodies in the composition are afucosylated or wherein fucosylation is undetectable in the composition.

Engineered glycoforms may be useful for a variety of purposes, including but not limited to enhancing or reducing effector function. Methods for generating engineered glycoforms in an antibody or antigen-binding fragment thereof described herein include but are not limited to those disclosed, e.g., in Umaña P et al., (1999) Nat Biotechnol 17: 176-180; Davies J et al., (2001) Biotechnol Bioeng 74: 288-294; Shields R L et al., (2002) J Biol Chem 277: 26733-26740; Shinkawa T et al., (2003) J Biol Chem 278: 3466-3473; Niwa R et al., (2004) Clin Cancer Res 1: 6248-6255; Presta L G et al., (2002) Biochem Soc Trans 30: 487-490; Kanda Y et al., (2007) Glycobiology 17: 104-118, U.S. Pat. Nos. 6,602,684; 6,946,292; and 7,214,775; U.S. Patent Publication Nos. US 2007/0248600; 2007/0178551; 2008/0060092; and 2006/0253928; International Publication Nos. WO 00/61739, WO 01/292246; WO 02/311140; and WO 02/30954; Potelligent™ technology (Biowa, Inc. Princeton, N.J.); and GlycoMAb® glycosylation engineering technology (Glycart biotechnology AG, Zurich, Switzerland). See also, e.g., Ferrara C et al., (2006) Biotechnol Bioeng 93: 851-861; International Publication Nos. WO 07/039818; WO 12/130831; WO 99/054342; WO 03/011878; and WO 04/065540.

In certain embodiments, any of the constant region mutations or modifications described herein can be introduced into one or both heavy chain constant regions of an antibody or antigen-binding fragment thereof described herein having two heavy chain constant regions.

In another particular embodiment, an antibody or antigen-binding fragment thereof described herein, which immunospecifically binds to B7-H4 (e.g., human B7-H4), comprises a heavy chain and a light chain, wherein (i) the heavy chain comprises a VH domain comprising the VH CDR1, VL CDR2, and VL CDR3 amino acid sequences of the 20502 antibody listed in Table 1; (ii) the light chain comprises a VL domain comprising the VL CDR1, VH CDR2, and VH CDR3 amino acid sequences of the 20502 antibody listed in Table 2; (iii) the heavy chain further comprises a constant heavy chain domain comprising the amino acid sequence of the constant domain of a human IgG, heavy chain; and (iv) the light chain further comprises a constant light chain domain comprising the amino acid sequence of the constant domain of a human kappa light chain.

In another particular embodiment, an antibody or antigen-binding fragment thereof described herein, which immunospecifically binds to B7-H4 (e.g., human B7-H4), comprises a heavy chain and a light chain, wherein (i) the heavy chain comprises a VH domain comprising the amino acid sequence of the VH domain of the 20502 antibody listed in Table 3; (ii) the light chain comprises a VL domain comprising the amino acid sequence of the VL domain of the 20502 antibody listed in Table 4; (iii) the heavy chain further comprises a constant heavy chain domain comprising the amino acid sequence of the constant domain of a human IgG, heavy chain; and (iv) the light chain further comprises a constant light chain domain comprising the amino acid sequence of the constant domain of a human kappa light chain.

In specific embodiments, an antibody or antigen-binding fragment thereof described herein, which immunospecifically binds to B7-H4 (e.g., human B7-H4) exhibits T cell checkpoint blockade activity. In specific embodiments, an antibody or antigen-binding fragment thereof described herein, which immunospecifically binds to B7-H4 (e.g., human B7-H4) increases interferon-gamma (IFNγ) production in T cells. In specific embodiments, an antibody or antigen-binding fragment thereof described herein, which immunospecifically binds to B7-H4 (e.g., human B7-H4) increases T cell proliferation. In specific embodiments, an antibody or antigen-binding fragment thereof described herein, which immunospecifically binds to B7-H4 (e.g., human B7-H4) increases CD4+ T cell proliferation. In specific embodiments, an antibody or antigen-binding fragment thereof described herein, which immunospecifically binds to B7-H4 (e.g., human B7-H4) increases CD8+ T cell proliferation.

In specific embodiments, an antibody or antigen-binding fragment thereof described herein, which immunospecifically binds to B7-H4 (e.g., human B7-H4) exhibits antibody-dependent cellular cytotoxicity (ADCC) activity. In specific embodiments, an antibody or antigen-binding fragment thereof described herein, which immunospecifically binds to B7-H4 (e.g., human B7-H4) exhibits antibody-dependent cellular cytotoxicity (ADCC) activity on cell lines with at least 300,000 cell surface B7-H4 molecules (e.g., SK-BR-3 cells). In specific embodiments, an antibody or antigen-binding fragment thereof described herein, which immunospecifically binds to B7-H4 (e.g., human B7-H4) exhibits antibody-dependent cellular cytotoxicity (ADCC) activity on cell lines with at least 100,000 cell surface B7-H4 molecules (e.g., HCC1569 cells). In specific embodiments, an antibody or antigen-binding fragment thereof described herein, which immunospecifically binds to B7-H4 (e.g., human B7-H4) exhibits antibody-dependent cellular cytotoxicity (ADCC) activity on cell lines with at least 50,000 cell surface B7-H4 molecules (e.g., ZR-75-1 cells). In specific embodiments, an antibody or antigen-binding fragment thereof described herein, which immunospecifically binds to B7-H4 (e.g., human B7-114) exhibits antibody-dependent cellular cytotoxicity (ADCC) activity on cell lines with at least 30,000 cell surface B7-H4 molecules (e.g., MDA-MB-468 cells). In specific embodiments, an antibody or antigen-binding fragment thereof described herein, which immunospecifically binds to B7-H4 (e.g., human B7-H4) exhibits antibody-dependent cellular cytotoxicity (ADCC) activity on cell lines with at least 15,000 cell surface B7-H4 molecules (e.g., HCC1964 cells).

In a specific aspect, an antigen-binding fragment as described herein, which immunospecifically binds to B7-H4 (e.g., human B7-H4), is selected from the group consisting of a Fab, Fab′, F(ab′)₂, and scFv, wherein the Fab, Fab′, F(ab′)₂, or scFv comprises a heavy chain variable region sequence and a light chain variable region sequence of an anti-B7-H4 antibody or antigen-binding fragment thereof as described herein. A Fab, Fab′, F(ab′)2, or scFv can be produced by any technique known to those of skill in the art. In certain embodiments, the Fab, Fab′, F(ab′)₂, or scFv further comprises a moiety that extends the half-life of the antibody in vivo. The moiety is also termed a “half-life extending moiety.” Any moiety known to those of skill in the art for extending the half-life of a Fab, Fab′, F(ab′)₂, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In certain embodiments, the Fab, Fab′, F(ab′)₂, or scFv can be modified by the addition of one or more C-terminal amino acids for attachment of the half-life extending moiety. In certain embodiments the half-life extending moiety is polyethylene glycol or human serum albumin. In certain embodiments, the Fab, Fab′, F(ab′)₂, or scFv is fused to an Fc region.

### 5.4 Pharmaceutical Compositions

Provided herein are methods of administering compositions comprising an anti-B7-H4 antibody or antigen-binding fragment thereof having the desired degree of purity in a physiologically acceptable carrier, excipient, or stabilizer (Remington's Pharmaceutical Sciences (1990) Mack Publishing Co., Easton, Pa.). Acceptable carriers, excipients, or stabilizers are nontoxic to recipients at the dosages and concentrations employed. (See, e.g., Gennaro, Remington. The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). The compositions to be used for in vivo administration can be sterile. This is readily accomplished by filtration through, e.g., sterile filtration membranes.

In some embodiments, methods of administering a pharmaceutical composition are provided, wherein the pharmaceutical composition comprises afucosylated anti-B7-H4 antibodies or antigen-binding fragments thereof and a pharmaceutically acceptable carrier. In specific embodiments, methods of administering a pharmaceutical composition are provided, wherein the pharmaceutical composition comprises afucosylated anti-B7-H4 antibodies or antigen-binding fragments e.g., wherein at least 80% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 85% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 90% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 95% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 96% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 97% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 98% of the antibodies in the composition are afucosylated. In specific embodiments, methods of administering a pharmaceutical composition are provided, wherein the pharmaceutical composition comprises afucosylated anti-B7-H4 antibodies or antigen-binding fragments e.g., wherein at least 99% of the antibodies in the composition are afucosylated. In specific embodiments, methods of administering a pharmaceutical composition are provided, wherein the pharmaceutical composition comprises afucosylated anti-B7-H4 antibodies or antigen-binding fragments wherein fucose is undetectable in the composition.

In some embodiments, methods of administering a pharmaceutical composition are provided, wherein the pharmaceutical composition comprises (i) an isolated antibody or antigen-binding fragment thereof that specifically binds to human B7-H4, comprising (a) the heavy chain variable region (VH) complementarity determining region (CDR) 1, VH CDR2, VH CDR3 and light chain variable region (VL) CDR1, CDR2, and CDR3 sequences of SEQ ID NOs:5-10, respectively. (b) a variable heavy chain region comprising the amino acid sequence of SEQ ID NO 1 and a variable light chain region comprising the amino acid sequence of SEQ ID NO:12, or (c) a heavy chain comprising the amino acid sequence of SEQ ID NO:21 and a light chain comprising the amino acid sequence of SEQ ID NO:22, and (ii) a pharmaceutically acceptable excipient.

Also provided herein is a method of administering a pharmaceutical composition, wherein the a pharmaceutical composition comprising (i) antibodies or antigen-binding fragments thereof that specifically bind to human B7-H4 and comprise the heavy chain variable region (VH) complementarity determining region (CDR) 1, VH CDR2, VH CDR3 and light chain variable region (VL) CDR1, CDR2, and CDR3 sequences of SEQ ID NOs:5-10, respectively and (ii) a pharmaceutically acceptable excipient, wherein at least 80%, at least 85%, at least 90%, at least 95%, at least 96%, at least 97%, at least 98%, or at least 99% of the antibodies or antigen-binding fragments thereof in the composition are afucosylated. In one embodiment, (i) the antibody or antigen-binding fragment thereof comprises a variable heavy chain region comprising the amino acid sequence of SEQ ID NO:11 and a variable light chain region comprising the amino acid sequence of SEQ ID NO:12 or (ii) the antibody comprises a heavy chain comprising the amino acid sequence of SEQ ID NO:21 and a light chain comprising the amino acid sequence of SEQ ID NO:22.

### 5.5 Antibody Production and Polynucleotides

Antibodies and antigen-binding fragments thereof that immunospecifically bind to B7-H4 (e.g., human B7-H4) can be produced by any method known in the art for the synthesis of antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J e/al., (2001) Molecular Cloning: A Laboratory Manual, Cold Spring Harbor Laboratory Press, Cold Spring Harbor, N.Y.; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

In certain aspects, provided herein are methods of administering an anti-B7-H4 antibody or antigen-binding fragment thereof or a pharmaceutical composition comprising such antibodies or fragments, wherein the antibodies or fragments are produced by recombinant expression of a polynucleotide comprising a nucleotide sequence in a host cell.

In certain aspects, the anti-B7-H4 antibody or antigen-binding fragment administered according to the methods provided herein comprises a heavy chain variable region encoded by a polynucleotide comprising the nucleotide sequence shown in Table 9 (i.e. SEQ ID NO:27). In certain aspects, the anti-B7-H4 antibody or antigen-binding fragment administered according to the methods provided herein comprises a heavy chain variable region encoded by a polynucleotide comprising the nucleotide sequence shown in Table 9 (i.e. SEQ ID NO:27) and a nucleotide sequence encoding a human gamma (γ) heavy chain constant region. In certain aspects, the anti-B7-H4 antibody or antigen-binding fragment administered according to the methods provided herein comprises a heavy chain variable region encoded by a polynucleotide comprising the nucleotide sequence shown in Table 9 (i.e. SEQ ID NO-27) and a heavy chain constant domain encoded by a polynucleotide comprising the nucleotide sequence of SEQ ID NO:26.

TABLE 9
Heavy chain variable region-encoding
polynucleotide sequences

| Anti- | Heavy Chain Variable Region-Encoding |
|---|---|
| body | Polynucleotide Sequence (SEQ ID NO) |
| 20502 | CAGCTGCAGCTGCAGGAGTCGGGCCCAGGACTGGTGAAGCCTTC |
| | GGAGACCCTGTCCCTCACCTGCACTGTCTCTGGTGGCTCCATCA |
| | AAAGTGGTAGTTACTACTGGGGCTGGATCCGCCAGCCCCCAGGG |
| | AAGGGGCTGGAGTGGATTGGGAACATCTATTATAGTGGGAGCAC |
| | CTACTACAACCCGTCCCTCAGAAGTCGAGTCACCATATCCGTAG |
| | ACACGTCCAAGAACCAGTTCTCCCTGAAGCTGAGTTCTGTGACC |
| | GCCGCAGACACGGCGGTGTACTACTGCGCCAGAGAAGGATCTTA |
| | CCCCAATCAGTTTGATCCATGGGGACAGGGTACATTGGTCACCG |
| | TCTCCTCA (SEQ ID NO: 27) |

In certain aspects, the anti-B7-H4 antibody or antigen-binding fragment administered according to the methods provided herein comprises a light chain variable region encoded by a polynucleotide comprising the nucleotide sequence shown in Table 10 (i.e., SEQ ID NO:28). In certain aspects, the anti-B7-H4 antibody or antigen-binding fragment administered according to the methods provided herein comprises a light chain variable region encoded by a polynucleotide comprising the nucleotide sequence shown in Table 10 (i.e. SEQ ID NO:28) and a nucleotide sequence encoding a human lambda light chain constant region. In certain aspects, the anti-B7-H4 antibody or antigen-binding fragment administered according to the methods provided herein comprises a light chain variable region encoded by a polynucleotide comprising the nucleotide sequence shown in Table 10 (i.e., SEQ ID NO:28) and a light chain constant domain encoded by a polynucleotide comprising the nucleotide sequence of SEQ ID NO:24

TABLE 10
Light chain variable region-encoding
polynuciectide sequences

| Anti- | Light Chain Variable Region-Encoding |
|---|---|
| body | Polynucleotide Sequence (SEQ ID NO) |
| 20502 | GAAATAGTGATGACGCAGTCTCCAGCCACCCTGTCTGTGTCT |
| | CCAGGGGAAAGAGCCACCCTCTCCTGCAGGGCCAGTCAGAGT |
| | GTTAGCAGCAACTTAGCCTGGTACCAGCAGAAACCTGGCCAG |
| | GCTCCCAGGCTCCTCATCTATGGTGCATCCACCAGGGCCACT |
| | GGTATCCCAGCCAGGTTCAGTGGCAGTGGGTCTGGGACAGAG |
| | TTCACTCTCACCATCAGCAGCCTGCAGTCTGAAGATTTTGCA |
| | GTTTATTACTGTCAGCAGTACCACTCCTTCCCTTTCACTTTT |
| | GGCGGAGGGACCAAGGTTGAGATCAAA (SEQ ID NO: 28) |

In certain aspects, the anti-B7-H4 antibody or antigen-binding fragment administered according to the methods provided herein comprises a variable heavy chain encoded by a polynucleotide comprising the variable heavy chain-encoding nucleotide sequence shown in Table 9 (i.e. SEQ ID NO:27) and a variable light chain encoded by a polynucleotide comprising the variable light chain-encoding nucleotide sequence shown in Table 10 (i.e., SEQ ID NO:28).

In certain aspects, the anti-B7-H4 antibody or antigen-binding fragment administered according to the methods provided herein comprises (i) a heavy chain encoded by a polynucleotide comprising the variable heavy chain-encoding nucleotide sequence shown in Table 9 (i.e. SEQ ID NO:27) and a nucleotide sequence encoding a human gamma (7) heavy chain constant region and (ii) a light chain encoded by a polynucleotide comprising the variable light chain-encoding nucleotide sequence shown in Table 10 (i.e. SEQ ID NO:28) and a nucleotide sequence encoding a human lambda light chain constant region.

In certain aspects, the anti-B7-H4 antibody or antigen-binding fragment administered according to the methods provided herein comprises (i) a heavy chain encoded by a polynucleotide comprising the variable heavy chain-encoding nucleotide sequence shown in Table 9 (i.e. SEQ ID NO:27) and the heavy chain constant domain-encoding nucleotide sequence of SEQ ID NO:26 and (ii) a light chain encoded by a polynucleotide comprising the variable light chain-encoding nucleotide sequence shown in Table 10 (i.e., SEQ ID NO:28) and the light chain constant domain-encoding nucleotide sequence of SEQ ID NO:24.

In certain aspects, the anti-B7-H4 antibodies or antigen-binding fragments administered according to the methods provided herein are encoded by polynucleotides encoding anti-B7-H4 antibodies or antigen-binding fragments thereof or a domain thereof that are optimized, e.g., by codon/RNA optimization, replacement with heterologous signal sequences, and elimination of mRNA instability elements. Methods to generate optimized nucleic acids encoding an anti-B7-H4 antibody or antigen-binding fragment thereof or a domain thereof (e.g., heavy chain, light chain, VH domain, or VL domain) for recombinant expression by introducing codon changes (e.g., a codon change that encodes the same amino acid due to the degeneracy of the genetic code) and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly.

Polynucleotides can be, e.g., in the form of RNA or in the form of DNA. DNA includes cDNA, genomic DNA, and synthetic DNA. DNA can be double-stranded or single-stranded. If single stranded, DNA can be the coding strand or non-coding (anti-sense) strand. In certain embodiments, the polynucleotide is a cDNA or a DNA lacking one or more introns. In certain embodiments, a polynucleotide is a non-naturally occurring polynucleotide. In certain embodiments, a polynucleotide is recombinantly produced. In certain embodiments, the polynucleotides are isolated. In certain embodiments, the polynucleotides are substantially pure. In certain embodiments, a polynucleotide is purified from natural components.

In certain aspects, vectors (e.g., expression vectors) comprise nucleotide sequences encoding anti-B7-H4 antibodies and antigen-binding fragments thereof or a domain thereof for recombinant expression in host cells, preferably in mammalian cells. In certain aspects, cells, e.g. host cells, comprise such vectors for recombinantly expressing anti-B7-H4 antibodies or antigen-binding fragments thereof described herein (e.g., human or humanized antibodies or antigen-binding fragments thereof). Thus, a method for producing an antibody or antigen-binding fragment thereof described herein can comprise expressing such antibody or antigen-binding fragment thereof in a host cell.

An expression vector can be transferred to a cell (e.g., host cell) by conventional techniques and the resulting cells can then be cultured by conventional techniques to produce an antibody or antigen-binding fragment thereof described herein (e.g., an antibody or antigen-binding fragment thereof comprising the six CDRs, the VH, the VL, the VH and the VL, the heavy chain, the light chain, or the heavy and the light chain of 20502) or a domain thereof (e.g., the VH, the VL, the VH and the VL, the heavy chain, or the light chain of 20502).

In certain embodiments, anti-B7-H4 antibodies or antigen-binding fragment thereof (e.g., an antibody or antigen-binding fragment thereof comprising the CDRs of 20502) administered according to the methods provided herein are produced in Potelligent® CHOK1SV cells.

In some embodiments, anti-B7-H4 antibodies or antigen-binding fragments thereof (e.g., an antibody or antigen-binding fragment thereof comprising the CDRs of 20502) administered according to the methods provided herein are produced in a host cell that lacks a functional alpha-1,6-fucosyltransferase gene (FUT8) gene. In some embodiments, the host cell is a CHO cell.

In specific embodiments, an antibody or antigen-binding fragment thereof administered according to the methods provided herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in a particular embodiment, a preparation of an antibody or antigen-binding fragment thereof described herein is substantially free of cellular material and/or chemical precursors.

The following examples are offered by way of illustration and not by way of limitation.

### 6. EXAMPLES

The examples in this Section (i.e., Section 6) are offered by way of illustration, and not by way of limitation.

### 6.1 Example 1: Assessment of Prevalence of B7-H4 Expression in Multiple Indications

The B7-H4 mouse monoclonal antibody A57.1 (ATCC Catalog No. PTA-5180) was used to detect the presence of B7-H4 on archival samples, a mixture of whole sections, and tumor microarrays. The samples were treated with the primary antibody and detected using a polymer detection system attached to DAB (Ventana Medical Systems).

B7-H4 was readily detected in the membrane and the cytosol in tumor tissues harvested from a variety of cancer patients, including invasive ductal carcinoma, triple negative breast cancer, ovarian cancer, non-small cell lung cancer and endometrial cancer. Moreover, frequency of expression was also high in the indications listed in Table 11.

TABLE 11
B7-H4 detection in tumors

| | | | Percent |
|---|---|---|---|
| Tumor Type | #Total | #Positive | Positive |
| Triple Negative Breast Cancer | 74 | 58 | 78% |
| Invasive Ductal Carcinoma | 51 | 38 | 74.50% |
| Endometrial Carcinoma | 77 | 54 | 70% |
| Ovarian Cancer | 141 | 85 | 50% |
| Non-Small Cell Lung Cancer | 47 | 19 | 40% |
| (Squamous) | | | |

B7-H4 is expressed in other cancers, such as breast cancer, kidney cancer (e.g., renal cell carcinoma), bladder cancer (e.g., urothelial cell carcinoma), pancreatic cancer, and thyroid cancer. See e.g., Zhu, J., et al., Asian Pacific J. Cancer Prev. 14: 3011-3015 (2011), Krambeck A, et al., PNAS 103: 10391-10396 (2006), Fan, M. et al., Int. J. Clin. Exp. Pathol. 7:6768-6775 (2014), Xu, H., et al., Oncology Letters 11: 1841-1846 (2016), and Liu, W., et al., Oncology Letters 8: 2527-2534 (2014).

### 6.2 Example 2: Afucosylated and Fucosylated 20502 Antibodies

Antibodies with Fc regions having reduced fucose content in glycan moieties may exhibit higher ADCC activity compared to a fully fucosylated antibody (Niwa R et al., Clinical Cancer Research 11(6):2327-36 (2005)). B7-H4 antibodies were generated in CHO-x cells (Yamane-Ohnuki N, et al. Biotechnology and Bioengineering 87(5): 614-22 (2004)) to produce normally fucosylated antibodies and in a CHO cell line engineered to produce afucosylated antibodies (CHO-y cells) (id.).

The fucosylated and afucosylated 20502 antibodies were characterized by surface plasmon resonance (SPR). Briefly, anti-human Fab antibody was immobilized on a carboxyl-derivatized SPR chip surface, and anti-B7-H4 antibodies were captured on the resulting surface at 5 ug/ml for 30 seconds. B7-H4 IgV-huIgG1 at various concentrations (0 nM, 3.7 nM, 11.1 nM, 33.3 nM, 100 nM, and 300 nM) was then flowed over the surface and allowed to bind to the anti-B7-H4 antibodies during the association phase, followed by a buffer wash during the dissociation phase.

B7-H4 IgV-huIgG1:
(SEQ ID NO: 29)
MASLGQILFWSIISIIIILAGAIALIIGFGISGRHSITVTTVASAGNIGED
GILSCTFEPDIKLSDIVIQWLKEGVLGLVHEFKEGKDELSEQDEMERGRTA
VFAQVIVGNASLRLKNVQLTDAGTYKCYIITSKGKGNANLEYKTGAFSGSE
PKSSDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQG
NVFSCSVMHEALHNHYTQKSLSLSPGK

Data was fitted using a 1:1 binding model, and the fucosylated and afucosylated 20502 showed similar binding to human B7-H4 protein. Thus, there is no impact of the glycosylation on binding.

The binding affinities of the Fc regions of fucosylated 20502 (Ab-F) and afucosylated 20502 (Ab-A) to FcγRIIIa (V158) were also characterized by surface plasmon resonance (SPR). Briefly, Protein A was covalently attached to a dextran chip using the amine coupling kit with 100 mM ethylenediamine in 100 mM Sodium Borate buffer, pH 8.0 as the blocking reagent. Ab-A or Ab-F was captured at 2 densities on separate flow cells, and a Protein A derivatized flow served as a reference control. Fc gamma RIIIA (V158) was diluted in HBS-P+ running buffer and injected at 6 concentrations (0 nM, 1.37 nM, 12.3 nM, 37 nM, 111 nM, 333 nM, and 1000 nM) in duplicate. The association constant, dissociation constant, and affinity for Ab-A binding were calculated using the Biacore T200 Evaluation Software 1:1 binding model. The affinity constant for Ab-A and Ab-F binding were determined using the Biacore T200 Evaluation Software steady state affinity model. The afucosylated B7-H4 antibody has a 140-fold higher affinity for Fc gamma receptor IIIA (V158) than the same antibody with a fucosylated Fc (Ab-F) (Table 12).

TABLE 12
Fcγ receptor IIIa (FcγRIIIa) V158 allele binding

| | ka (1/Ms) | kd (1/s) | K_{D} (nM) |
|---|---|---|---|
| Ab-A | 6.46E+05 | 9.54E−10 | 15 |
| Ab-F | N/A | N/A | 210 |

The T cell checkpoint blockade activity of fucosylated and afucosylated 20502 antibodies were also characterized. In these experiments, primary human T cells were enriched from PBMCs using the EasySep™ Human T Cell Enrichment Kit based on the manufacturer's instructions. Enriched T cells were incubated at 2×10⁵ cell/mL with anti-CD3/anti-CD28 Dynabeads, at a one bead per cell ratio, at 37° C. Six days later, the beads were magnetically removed, and T cells were washed and incubated at 1×10⁶ cell/mL with 10 U/mL IL-2 at 37° C. Four days later, T cells were washed and incubated at 1×10⁶ cells/mL along with artificial antigen presenting cells (aAPCs) at a 2×10⁶ cells/mL concentration at 37° C. in the presence of B7-H4 antibody dose titration. aAPCs were treated with Mitomycin C for one hour at 37° C. and then thoroughly washed prior to adding to the T cell co-culture. 72 hours after co-culture of T cells, aAPCs, and B7-H4 antibodies, plates were centrifuged and supernatants were harvested and assessed for IFNγ production by ELISA. IFNγ production was plotted vs. antibody concentration and the EC50 potency was calculated using nonlinear regression curve fit (GraphPad Prism).

The B7-H4 antibodies demonstrated potent T cell checkpoint blockade activity as measured by an increase in IFNγ production. Moreover, there was no demonstrable difference in potency between afucosylated and fucosylated antibodies (Table 13.)

TABLE 13
T Cell Checkpoint blockade potency

aAPC Assay (EC50 +/− STD; nM)

| Antibody | BIN | Afucosylated | Fucosylated |
|---|---|---|---|
| 20502 | 3 | 0.89 +/− 0.44 | 0.74 +/− 0.39 |

In additional experiments, the ADCC activity of fucosylated and afucosylated 20502 antibodies was also characterized against a B7-H4-expressing target cell line. Specifically, primary human PBMCs cells were cytokine activated at 1×10⁶ cells/mL with 200 IU/mL IL-2 at 37° C. The next day, cells were washed and incubated at a 40:1 Effector:Target ratio with SK-BR-3 target cells that were labeled with Calcein-AM. 4 hours after incubation, target cell lysis was quantified using a fluorimeter. A Triton/X treated sample served as the max lysis control sample, whereas a media alone treated sample served as the background lysis control sample. The percent (%) specific lysis was calculated as follows: [1−((sample−media control)/(max lysis−media control))]×100. The percent (%) specific lysis was plotted vs. antibody concentration and the EC50 potency was calculated using nonlinear regression curve fit (GraphPad Prism).

The B7-H4 antibodies demonstrated potent dose-dependent ADCC activity against the endogenous B7-H4 expressing breast cell line SK-BR-3. Moreover, the afucosylated antibodies demonstrated significantly more potent ADCC activity in comparison to the fucosylated antibodies (Table 14).

TABLE 14
ADCC activity

| | | ADCC Assay (EC50 +/− STD; nM) | |
|---|---|---|---|
| Antibody | BIN | Afucosylated | Fucosylated |
| 20502 | 3 | 0.0007 +/− 1.1 × 10E−3 | 0.0370 +/− 6.2E−2 |

### 6.3 Example 3: Correlation of ADCC Activity with Receptor Density

B7-H4 density was quantified on the surface of SK-BR-3, HCC1569, ZR-75-1, MDA-MB-48, and HCC1964 cells by FACS according to the manufacturer's specifications. Specifically, 1×10⁵ cells were incubated with 15 μg/mL B7-H4 antibody on ice for 25 minutes. In parallel, one drop of Quantum™ Simply Cellular (QSC) microspheres (pre-coated with increasing concentrations of anti-mouse IgG capture antibody) was also incubated with 15 ug/mL B7-H4 antibody on ice for 25 minutes. Following incubation, cells and QSC microspheres were pelleted and washed, and samples were acquired on a flow cytometer. Data was analyzed using the FlowJo software. Mean fluorescence intensity (MFI) was calculated and entered into the QuickCal® spreadsheet. A regression associating each bead's fluorescence channel value to its pre-assigned Antibody Binding Capacity (ABC) value will be calculated automatically. An ABC value was assigned once the MFI values for the labeled cells are also added into the template).

B7-H4 antibodies were assessed for ADCC activity against B7-H4 expressing target cell lines with different levels of B7-H4 cell surface density. Specifically, 1×10⁴ SK-BR-3, HCC1569, ZR-75-1, MDA-MB-468, or HCC1964 target cells were co-incubated with dose-titrations of B7-H4 antibody at 4° C. 25 minutes later, a single use vial of Jurkat-huCD16 reporter cells from Promega was thawed, and 7.5×10⁴ cells were added to the target cell/B7-H4 antibody mixture and incubated at 37° C. 24 hours later, the samples were brought to room temperature (RT) and incubated with Bio-Glo buffer. The substrate and luminescence were quantified on an EnVision multi-label reader. The data was plotted as luminescence vs. antibody concentration and the EC50 potency was calculated using nonlinear regression curve fit (GraphPad Prism).

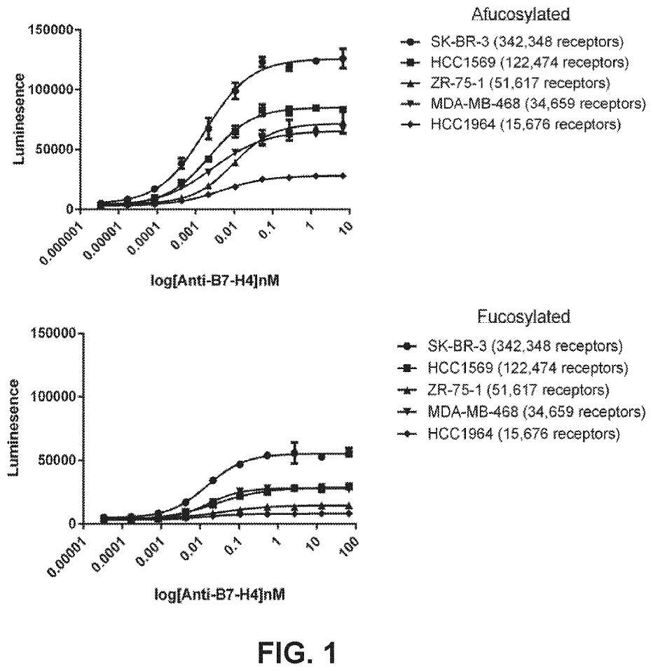

B7-H4 antibody ADCC activity was dependent on B7-H4 cell surface density: as the numbers of cell surface molecules decreased, the amount of maximal ADCC activity also decreased. Moreover, afucosylated antibodies demonstrated improved ADCC activity in comparison to the fucosylated antibodies, especially against target cells with lower levels of B7-H4 cell surface density (FIG. 1).

### 6.4 Example 4: In Vivo Anti-Tumor Efficacy

Unlike human tumors, mouse models do not endogenously express high levels of B7-H4 protein. To test afucosylated 20502 in mice, syngeneic mouse cancer models using murine tumor cell lines engineered to express B7-H4 protein were used. Seven week old female BALB/c mice were purchased from Charles River Laboratories (Hollister, Calif.) and were acclimated for up to three weeks before the start of the studies. The murine colorectal carcinoma cell line CT26 was engineered to express a chimeric protein consisting of the extracellular domain of murine B7-H4 with the transmembrane domain of murine B7H3. These tumor cells were implanted subcutaneously over the right flank of the mice at 1.0×10⁶ cells/200 μL/mouse. Prior to inoculation, the cells were cultured for no more than three passages in RPMI 1640 medium supplemented with 10% heat-inactivated Fetal Bovine Serum (FBS), 2 mM L-Glutamine. Cells were grown at 37° C. in a humidified atmosphere with 5% CO₂. Upon reaching 80-85% confluence, cells were harvested and resuspended in a 1:1 mixture of serum-free RPMI 1640 and Matrigel at 5×10⁶ cells per milliliter).

Mice were monitored twice weekly following cell implantation for tumor growth. For tumor measurements, the length and width of each tumor was measured using calipers and volume was calculated according to the formula: tumor volume (mm³)=(width (mm)×length (mm²)/2. On the day of treatment initiation, all tumors were measured, outliers were excluded, and mice were randomly assigned to treatment groups. For anti-B7-H4 treatment, afucosylated 20502 antibodies were administered. As controls, mice were administered polyclonal human IgG (Bio X Cell, BE0092) or mouse IgG2a (Bio X Cell, BE0085). The antibodies were administered four times via intravenous (i.v.) injection twice weekly beginning on Day 4 or 5 after inoculation.

Tumors continued to be measured at least twice per week until tumor volume exceeded 10% of animal weight, or approximately 2000 mm³. The change in tumor size is shown by graphing individual tumors relative to the day upon which animals were inoculated with CT26 cells. P-values were calculated using unpaired, two-tailed t-test analyses of the calculated tumor volumes on each day of the study.

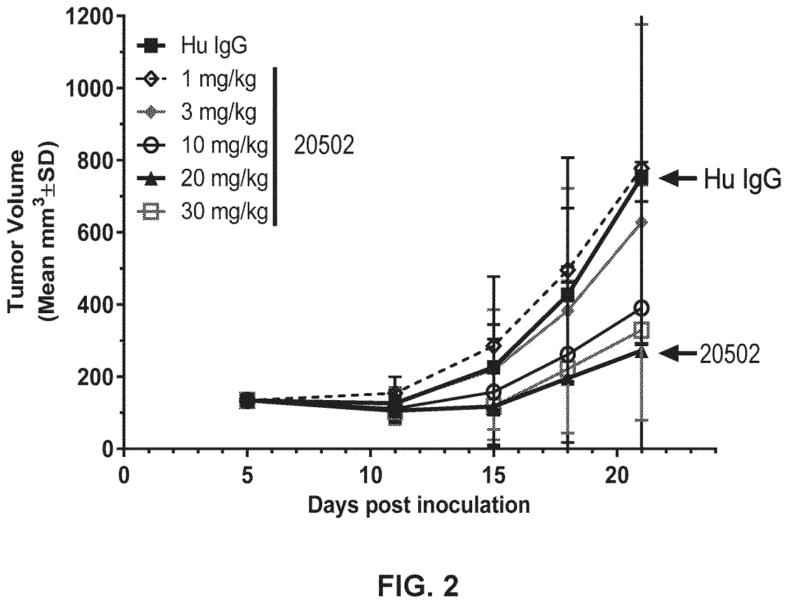

The engineered CT26 model expressing B7-H4 protein demonstrated significant dose-dependent tumor growth inhibition in 5 dose levels in the dose range from 1 to 30 mg/kg (FIG. 2). The most common impact in individual animals was tumor growth inhibition. However, afucosylated 20502 treatment did result in complete tumor regression in 7 of 15 mice in the 30 mg/kg group, 6 of 15 mice in the 20 mg/kg group, and 5 of 15 mice in the 10 mg/kg group (FIG. 2). Afucosylated 20502 dosed at 3 mg/kg or lower elicited minimal anti-tumor activity compared to the negative control treatment group (human IgG).

### 6.5 Example 5: Nonclinical Pharmacokinetics

The pharmacokinetics (PK) and toxicokinetics (TK) of afucosylated 20502 were evaluated following a single and/or repeat weekly intravenous (IV) administration in mice, rats, and cynomolgus monkeys. PK characteristics observed were consistent across all studies. In all species, afucosylated 20502 demonstrated linear PK and a dose proportional increase in exposure (area under serum concentration-time curve [AUC]) with increasing dose. There was an approximate 2-fold increase in weekly exposure (AUC_{0-7days}) following 4 weekly administrations of 20502 between first and last dose; however, steady state was not achieved. No substantial gender differences were apparent in the serum afucosylated 20502 concentration-time profiles. In the cynomolgus monkey (across 2 different studies), half-life estimated from recovery animals ranged from approximately 8.8 days to 12 days, with doses levels ranging from 1 to 100 mg/kg. The estimated half-life in rat following a single IV infusion administration at 40 mg/kg was approximately 13.2 days. The PK characteristics of afucosylated 20502 in animals support IV infusion in humans with a once every 3 week (Q3W) dose regimen.

### 6.6 Example 6: Toxicology

Toxicology studies with afucosylated 20502 were performed in rat and cynomolgus monkey. The studies included a pilot single dose pharmacokinetic (PK)/tolerability study in rats, a pilot repeat-dose toxicity study in cynomolgus monkeys, and investigational new drug (IND)-enabling Good Laboratory Practices (GLP) repeat-dose toxicity studies in rats and cynomolgus monkeys, as well as a GLP tissue cross-reactivity study with human, rat, and cynomolgus monkey tissues.

In the single dose pilot tolerability study in rats, the animals received doses up to 40 mg/kg as a 30-minute intravenous (IV) infusion. Afucosylated 20502 had no effect on clinical observations, body weights, food consumption, clinical pathology (serum chemistry or hematology) assessments, gross observations, organ weights, or histopathology assessment.

In the pilot repeat-dose toxicology study cynomolgus monkeys received 4 weekly IV doses of afucosylated 20502 up to 100 mg/kg as a 30-minute IV infusion. All doses were well tolerated by cynomolgus monkeys. There were no test article-related unscheduled mortalities or changes attributed to administration of afucosylated 20502 during assessment of clinical observations, body weights, clinical pathology, necropsy, organ weight, or histopathology parameters.

In the repeat-dose GLP toxicology studies, afucosylated 20502 was administered by IV at dose levels of 1, 10, and 100 mg/kg/dose to both rats and cynomolgus monkeys for 4 weekly doses. Reversibility of toxicity was evaluated during a 6-week recovery period following the final administration. Parameters for evaluation included ophthalmic examinations, clinical observations, body temperatures, body weights, food consumption, hematology, coagulation, clinical chemistry, urinalysis, organ weights, macroscopic, and microscopic evaluation. In the cynomolgus monkey study, electrocardiograms (ECGs) were also assessed to evaluate potential cardiac toxicities.

During the evaluation of the GLP rat study, afucosylated 20502 was generally well tolerated, and there were no toxic effects attributed to afucosylated 20502. The no-observed-adverse-effect level (NOAEL) in Sprague Dawley rats was considered to be 100 mg/kg/dose.

In the GLP cynomolgus monkey study, afucosylated 20502 was generally well tolerated, and there were no adverse events (AEs) attributed to afucosylated 20502 observed in any of the parameters evaluated. During the study, a higher incidence of diarrhea was observed at the end of the dosing phase in the higher dose groups. Due to the higher incidence of affected animals in the mid and high dose, as well as onset in the later phase of the dosing period, a relationship with afucosylated 20502 exposure is possible. There were no microscopic changes in the intestinal tract in animals treated with afucosylated 20502, including animals with diarrhea; therefore, this finding was considered non-adverse but possibly related to the test article. There was a single mortality in the study. One animal in the mid-dose recovery group was found dead on Study Day 35, 14 days post the last dose. Clinical observations, macroscopic and microscopic evaluation were consistent with the diagnosis of intestinal torsion. Intestinal torsions occasionally occur in cynomolgus monkeys, and this was considered a spontaneous condition in this animal and not test article-related. The NOAEL in cynomolgus monkey was considered to be 100 mg/kg/dose.

In addition to in vivo toxicology studies, a GLP-compliant tissue cross reactivity study was performed to compare the binding of afucosylated 20502 to a panel of 36 tissues from rat, cynomolgus monkey, and human. The results showed that the binding pattern of afucosylated 20502 was similar among the 3 species and limited to the mammary gland epithelium.

Thus, afucosylated 20502 was well tolerated in cynomolgus monkey and rat. The NOAEL in both species was considered to be 100 mg/kg/dose, the highest dose tested when given as 4 weekly IV doses.

### 6.7 Example 7: Phase 1a Afucosylated 20502 Dose Escalation and Exploration

A phase 1a open-label multicenter study is conducted in up to 34 patients with advanced solid tumors using afucosylated 20502.

(A) Study Design

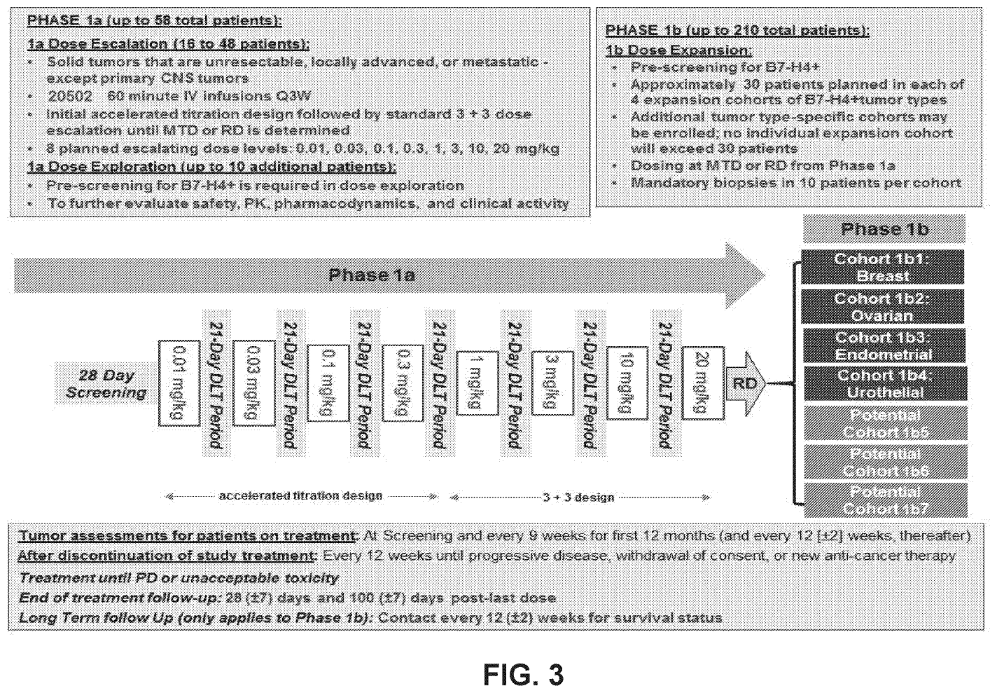

Phase 1a includes a Dose Escalation phase and a Dose Exploration phase. The Phase 1a study schema is provided in FIG. 3. In both the Dose Escalation and Dose Exploration phases, afucosylated 20502 is administered as a 60-minute intravenous (IV) infusion every three weeks (Q3W) on Day 1 of each 21-day cycle. The dose of afucosylated 20502 is based on body weight at Cycle 1 Day 1. After Cycle 1, the dose is recalculated at each infusion visit only if the patient's weight has changed >10% from Cycle 1, Day 1.

The Phase 1a Dose Escalation includes an initial accelerated titration design followed by a standard 3+3 dose escalation design at dose levels greater than or equal to 1 mg/kg until the maximum tolerated dose (MTD) and/or recommended dose (RD) for Phase 1b is determined. Up to 16 to 48 patients participate in Dose Escalation. Doses from 0.01 (or 0.005) to 20 mg/kg are administered per the cohorts outlined in Table 15, and patients' second doses must be at least 21 days after their first doses.

TABLE 15
Dose Levels

| Dose Level | Dose | Regimen |
|---|---|---|
| −1* | Afucosylated 20502 0.005 mg/kg | Q3W |
| 1 | Afucosylated 20502 0.01 mg/kg | Q3W |
| 2 | Afucosylated 20502 0.03 mg/kg | Q3W |
| 3 | Afucosylated 20502 0.1 mg/kg | Q3W |
| 4 | Afucosylated 20502 0.3 mg/kg | Q3W |
| 5 | Afucosylated 20502 1 mg/kg | Q3W |
| 6 | Afucosylated 20502 3 mg/kg | Q3W |
| 7 | Afucosylated 20502 10 mg/kg | Q3W |
| 8 | Afucosylated 20502 20 mg/kg | Q3W |

*If the MTD is exceeded at the first dose level of afucosylated 20502 (0.01 mg/kg), the dose will be reduced to 0.005 mg/kg.

During Phase 1a Dose Escalation, the Dose-Limiting Toxicity (DLT) evaluation begins on the first day of treatment upon start of infusion and continues for 21 days. A DLT is defined as any of the following regardless of attribution (except for those events clearly due to the underlying disease or extraneous causes): (i) Grade 3 or higher non-hematologic toxicity (other than Grade 3 nausea, vomiting and diarrhea) occurring with the first 21 days of treatment), (ii) Grade 3 nausea, vomiting and diarrhea lasting at least 72 hours despite optimal supportive care, occurring within first 21 days of treatment, (iii) febrile neutropenia and/or documented infection with absolute neutrophil count (ANC) less than 1.0×10⁹ per L, Grade 4 neutropenia lasting for more than 7 days, Grade 4 thrombocytopenia (less than 25.0-10⁹ per L), or Grade 3 thrombocytopenia (less than 50.0 25.0×10⁹ per L) accompanied by bleeding within first 21 days of treatment, (iv) aspartate aminotransferase/alanine transaminase (AST/ALT) more than 3 times the upper limit of normal (ULN) and concurrent total bilirubin more than twice ULN not related to liver involvement with cancer, (v) other Grade 3 laboratory values that are not of clinical significance that do not resolve within 72 hours, or (vi) any Grade 4 laboratory value regardless of clinical sequelae.

An accelerated titration design enrolling at least 1 patient at each dose level is carried out for dose levels 0.01, 0.03, 0.1 and 0.3 mg/kg. Dose escalation to the next dose level proceeds after at least 1 patient completes the 21-day evaluation interval. If a single patient experiences a DLT or at least 2 patients experience moderate AEs (at any dose level) during the 21-day evaluation interval, additional patients are enrolled at the current dose level, and standard 3+3 dose escalation criteria applies for that cohort as well as all subsequent dosing cohorts. Moderate AEs are defined as ≥Grade 2 AEs regardless of attribution (except for those events clearly due to the underlying disease or extraneous causes). Grade 2 laboratory values are not considered as moderate AEs for this purpose unless accompanied by clinical sequelae.

Intra-patient dose escalation will be permitted in patients enrolled at dose levels below 1 mg/kg provided: (i) the patient did not experience a DLT; (ii) all other AEs have recovered to Grade 1 or lower prior to dose escalation; (iii) the patient may only dose escalate by a maximum of 1 dose level every 21 days; and (iv) the patient cannot dose escalate beyond 1 mg/kg dose level unless the dose level has been cleared according to the standard 3+3 dose escalation design as described below.

The algorithm outlined in Table 16A below is used for all standard 3+3 dose escalations.

TABLE 16A
Phase 1a Algorithm for 3 + 3 Dose-Escalation Decisions

| Number of | |
|---|---|
| Patients | |
| with DLT at | |
| a Given Dose | |
| Level | Dose Escalation Decision Rule |
| 0/3 | Enroll 3 patients at next dose level (next/higher cohort) |
| 1/3 | Enroll 3 additional patients at current dose level |
| | (current cohort) |
| ≥2/3 | Stop enrollment. Enter 3 more patients at the previous |
| | dose level (previous/lower cohort), if only 3 were |
| | previously entered, or at an intermediate dose level |
| 1/6 | Enroll 3 patients at next, dose level (next/higher cohort) |
| ≥2/6 | Stop enrollment. Enter 3 more patients at the lower |
| | dose level (previous/lower cohort), if only 3 were |
| | previously entered, or at an intermediate dose level |

The MTD and/or RD of afucosylated 20502 for Phase 1a is identified based on an evaluation of the overall safety, tolerability, pharmacodynamics, pharmacokinetics, and preliminary efficacy. The RD will take into account toxicities observed both during and beyond the DLT evaluation period, as well as dose reductions and discontinuations due to toxicity that do not meet the DLT criteria. The RD, therefore, may or may not be the same as the identified MTD. For example, if the MTD is not reached, or if data from subsequent cycles of treatment from Phase 1a provide additional insight on the safety profile, then the RD may be a different, though not higher, dose than the MTD. The MTD will be at a dose level where no more than ⅙ patients reported a DLT. The RD will also be a dose where no more than ⅙ patients reported a DLT, but it may be lower than the MTD. In some embodiments, the MTD will be at a dose level where no more than ⅓, ¼, or ⅕ patients reported a DLT. The RD will also be a dose where no more than ⅓, ¼, or ⅕ patients reported a DLT, but it may be lower than the MTD.

The Phase 1a Dose Exploration cohort enrolls beyond 3 patients (up to 10 additional patients across all dose levels). Pre-screening of archival tumor tissue (or fresh biopsy if archival tissue is not available) is used to test for B7-H4 expression levels by immunohistochemistry (IHC) for all patients during Phase 1a Dose Exploration. Archival tumor tissue (or fresh biopsy) can be used for biomarker analysis, as herein. In addition, fresh biopsies are used during screening and post-treatment for expanded pharmacodynamics analysis.

In one embodiment, proposed dose cohorts for the Phase 1a Monotherapy Dose Exploration are shown in Table 16B.

TABLE 16B
Proposed Dose Cohort/Level for Phase 1a Dose Exploration

| | | |
|---|---|---|
| Cohort | Dose | Regimen |

| | | | |
|---|---|---|---|
| 1 | 3 | mg/kg | Q3W |
| 2 | 10 | mg/kg | Q3W |

| | | |
|---|---|---|
| 3 | MTD/RD | Q3W |

Abbreviations: MID = maximum tolerated dose; Q3W = once every 3 weeks; RD = recommended dose.

In one embodiment, the recommended dose is 20 mg/kg.

(B) Subjects

A total of 12 to 24 patients are identified based on the following inclusion and exclusion criteria.

Patients in Phase 1a meet all of the following inclusion criteria:
Histologically confirmed solid tumors except primary central nervous system (CNS) tumors;
Disease that is unresectable, locally advanced, or metastatic;
Refractory to or intolerant of existing therapies known to provide clinical benefit for patient's condition; and
At least one measurable lesion at baseline according to RECIST v1.1 (Response Evaluation Criteria in Solid Tumors 1.1); tumor sites situated in a previously irradiated area, or in an area subjected to other loco-reginal therapy, are not considered measurable unless there has been demonstrated progression in the lesion.

Exclusion criteria: Patients in Phase 1a do not have a history of anti-drug antibodies (ADAs), severe allergic, anaphylactic, or other infusion-related reaction to a previous biologic agent and do not have a known hypersensitivity to any component in the afucosylated 20502 formulation.

(C) Results

The incidence of Grade 3 and Grade 4 adverse events and clinical laboratory abnormalities defined as dose limiting toxicities are evaluated to show that afucosylated 20502 is safe and tolerable in patients with advanced solid tumors. The incidence of adverse events, clinical laboratory abnormalities and ECG abnormalities are evaluated to determine the maximum tolerated dose and/or recommended dose of afucosylated 20502.

Pharmacokinetic parameters (AUC (area under serum concentration time curve), Cₘₐₓ (maximum serum concentration), Cₘᵢₙ (minimum serum concentration), clearance (CL), t_{1/2} (terminal half-life), Vₛₛ (volume of distribution at a steady state), and C_{trough} (trough serum concentration at the end of a dose interval) in patients with advanced solid tumors are determined from serum afucosylated 20502 concentration-time data using a non-compartmental analysis. Serum afucosylated 20502 concentrations are determined using enzyme linked immunosorbent assay (ELISA) method. The impact of immunogenicity (i.e., anti-drug antibody immune responses to afucosylated 20502) in patients with advanced solid tumors on afucosylated 20502 exposure is assessed by measuring total anti-afucosylated 20502 antibodies from all patients.

The clinical benefits of afucosylated 20502 in patients with advanced solid tumors are also demonstrated. Tumor assessments include a clinical examination and imaging (e.g., computed tomography (CT) scans with appropriate slice thickness per RECIST v 1.1 or magnetic resonance imaging (MRI)). Tumors are assessed at screening, every 9 weeks for the first 12 months, and every 12 weeks (+/−2 weeks) thereafter to show inhibition of tumor growth and tumor regression (e.g., complete tumor regression).

The overall response rate (ORR), duration of response (DOR), and progression-free survival (PFS) are also determined as measurements of efficacy. The ORR is defined as the total number of patients with confirmed responses (either complete response (CR) or partial response (PR) per RECIST v.1.1) divided by the total number of patients who are evaluable for a response. The DOR is defined as the time from onset of response (CR or PR) that is subsequently confirmed to the first observation of progressive disease or death due to any cause. PFS is defined as the time from the patient's first dose to the first observation of progressive disease or death due to any cause.

Pharmacodynamic biomarkers are also observed. An analysis can be performed of the immune cell infiltrate in pre-treatment and on-treatment tumor biopsies. For example, changes in markers of tumor immune infiltrate (including but not limited to natural killer cells (NK), CD4, CD8, and/or other select immune biomarkers) are assessed by IHC and/or ribonucleic acid (RNA) analysis. In addition, changes in cytokine levels (e.g., IL-2, IL-6, IL-10, TNF, and/or interferon gamma (IFNγ)) are assessed by multiplex analysis.

Patients received afucosylated 20502 across a range of dose levels. 24 patients who were unselected for B7-H4 with advanced solid tumors and with a median of three (3) prior therapies were treated with afucosylated 20502 antibody. In a dose escalation cohort, 18 patients received dose levels from 0.01 mg/kg to 20 mg/kg every three weeks (Q3W) in an accelerated titration, followed by 3+3 design. Patients were treated with a median number of 3 (range=1-11) doses of afucosylated 20502. Most received either 3 mg/kg (n=8) or 10 mg/kg (n=6) afucosylated 20502. Seven (7) of the patients from the dose escalation cohort were retrospectively identified as B7-H4 positive. In a separate dose exploration cohort, six (6) B7-H4 positive patients (out of the total 24 patients) were treated at doses of 3 mg/kg or 10 mg/kg Q3W with mandatory pre- and on-treatment biopsies. No dose reductions were required, and no dose-limiting toxicities or treatment-related serious adverse events (SAEs) were observed in 24 patients Thus, afucosylated 20502 demonstrated a favorable safety profile, and the data suggest that 20 mg/kg can be selected as the recommended dose.

### 6.8 Example 8: Phase 1b Afucosylated 20502 Dose Expansion

A phase 1b open-label multicenter study is conducted using afucosylated 20502 in up to 210 patients with specific solid tumor types with B7-H4 expression levels determined by immunohistochemistry (IHC). The specific solid tumor types were identified based on their high prevalence of B7-H4 expression and limited availability of effective therapies in the unresectable and metastatic setting.

(A) Study Design

Phase 1b is a dose expansion portion of the study. The Phase 1b study schema is provided in FIG. 3. Enrollment into Phase 1b Dose Expansion begins after identification of the maximum tolerated dose (MTD) and/or recommended dose (RD) in Phase 1a.

Phase 1b includes tumor-specific cohorts of up to 30 patients each as shown in Table 17. The phase 1b study may have more or fewer cohorts than shown in Table 17, but not to exceed 7 cohorts.

TABLE 17
Phase 1b Expansion Cohorts and Tumor Types

| Cohort | Tumor Type |
|---|---|
| 1b1 | Breast cancer |
| 1b2 | Ovarian cancer |
| 1b3 | Endometrial cancer |
| 1b4 | Urothelial cancer |

Archival tumor tissue (or fresh biopsy if archival tissue is not available) is used to test for B7-H4 expression levels by immunohistochemistry (IHC) for pre-screening all patients and for biomarker analysis. In addition, fresh biopsies, taken during screening and post-treatment, are used for expanded pharmacodynamic analysis from a subset of patients (10 patients per 30 patient cohort).

Afucosylated 20502 is administered as 60-minute intravenous (IV) dose every three weeks (Q3W) on Day 1 of each 21-day cycle. The dose of afucosylated 20502 is based on body weight at Cycle 1 Day 1. After Cycle 1, the dose will be recalculated at each infusion visit only if the patient's weight has changed >10% from Cycle 1, Day 1.

(B) Subjects

Up to 30 patients with breast cancer, ovarian cancer, endometrial cancer, or urothelial cancer will participate. Additional tumor type-specific cohorts of up to 30 patients each may also participate.

Patients in Phase 1b meet all of the following inclusion criteria
All inclusion criteria for Phase 1a (histologically confirmed solid tumors except primary central nervous system (CNS) tumors);
Positive for B7-H4 expression in an archival or fresh tumor sample as evaluated by an immunohistochemistry (IHC) assay;
For Cohort 1b1—breast cancer
   Histologically or cytologically confirmed metastatic breast cancer,
   Progressive disease on or after or unable to tolerate anthracycline and taxane chemotherapy;
   Progressive disease on or after at least one systemic chemotherapy in the metastatic setting;
   Patients who have estrogen receptor (ER) and/or progesterone receptor (PR) positive disease (defined as ER and/or PR>1%) must be hormone refractory (progressed after 3 sequential endocrine therapies) or have symptomatic visceral disease, and
   Patients have HER2—negative disease,
For triple negative breast cancer (TNBC) patients in Cohort 1b1:
   Histologically or cytologically confirmed metastatic TNBC; and
   At least two prior lines of systemic chemotherapy with at least one being administered in the metastatic setting;
For hormone receptor positive (HR+) breast cancer patients in Cohort 1b1:
   Histologically or cytologically confirmed metastatic HR+ breast carcinoma,
   Patients have received at least two prior lines of hormonal therapy; and
   Patients have received at least one prior line of systemic chemotherapy (in the adjuvant or metastatic setting)
For Cohort 1b2—ovarian cancer
   Histologically or cytologically confirmed diagnosis of recurrent epithelial ovarian, primary peritoneal, or fallopian tube cancer that is refractory to existing therapies known to provide clinical benefit; and
   Progressive disease on or after at least two prior regimens of treatment including at least one platinum-containing regimen, or unable to tolerate additional chemotherapy;
For Cohort 1b3—endometrial cancer
   Histologically or cytologically confirmed recurrent or persistent endometrial cancer that is refractory to curative or established treatments; and
   Progressive disease on or after at least one prior regimen of systemic chemotherapy, or unable to tolerate systemic chemotherapy;
For Cohort b4—urothelial cancer
   Histologically or cytologically confirmed urothelial cancer; and
   Progressive disease on or after or intolerance to a platinum-containing regimen and a PD-1/PD-L1 directed agent.

Patients in Phase 1b do not have a history of anti-drug antibodies (ADAs), severe allergic, anaphylactic, or other infusion-related reaction to a previous biologic agent and do not have a known hypersensitivity to any component in the afucosylated 20502 formulation.

(C) Results

The incidence of adverse events, clinical laboratory abnormalities and ECG abnormalities are evaluated to demonstrate the safety and tolerability of afucosylated 20502 in patients with B7-H4-positive advanced solid tumors.

Pharmacokinetic parameters (AUC, Cₘₐₓ, Cₘᵢₙ, CL, t_{1/2}, Vₛₛ (volume of distribution at a steady state)) in patients with B7-H4-positive advanced solid tumors are determined from serum afucosylated 20502 concentration-time data using a non-compartmental analysis Serum afucosylated 20502 concentrations are determined using enzyme linked immunosorbent assay (ELISA) method.

Pharmacodynamic biomarkers are also observed. For example, changes in markers of tumor immune infiltrate (including, but not limited to, natural killer cells (NK), CD4, CD8, and/or other select immune biomarkers) are assessed by HC and/or ribonucleic acid (RNA) analysis. In addition, changes in cytokine levels (e.g., IL-2, IL-6, IL-10, TNF, and/or interferon gamma (IFNγ)) are assessed by multiplex analysis.

The impact of immunogenicity (i.e., anti-drug antibody immune responses to afucosylated 20502) in patients with B7-H4-positive advanced solid tumors on afucosylated 20502 exposure is assessed by measuring total anti-afucosylated 20502 antibodies from all patients.

The clinical benefits of afucosylated 20502 are also demonstrated. Tumors assessments include a clinical examination and imaging (e.g., computed tomography (CT) scans with appropriate slice thickness per RECIST v 1.1 or magnetic resonance imaging (MRI)). Tumors are assessed at screening, every 9 weeks for the first 12 months, and every 12 weeks (+/−2 weeks) thereafter to show inhibition of tumor growth and tumor regression (e.g., complete tumor regression).

The overall survival, defined as time from a patient's first dose to death due to any cause is also determined as a measure of efficacy. The overall survival rates demonstrate the clinical benefit of afucosylated 20502 in patients with B7-H4-positive advanced solid tumors.

The invention is not to be limited in scope by the specific embodiments described herein. Indeed, various modifications of the invention in addition to those described will become apparent to those skilled in the art from the foregoing description and accompanying figures Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1. A method of treating a solid tumor in a human subject, the method comprising administering to the subject about 0.005 to about 20 mg/kg of an antibody or antigen-binding fragment thereof that specifically binds to human B7-H4 and comprises a heavy chain variable region (VH) complementarity determining region (CDR) 1 comprising the amino acid sequence of SEQ ID NO:5, a VH CDR2 comprising the amino acid sequence of SEQ ID NO:6, a VH CDR3 comprising the amino acid sequence of SEQ ID NO:7, a light chain variable region (VL) CDR1 comprising the amino acid sequence of SEQ ID NO:8, a VL CDR2 comprising the amino acid sequence of SEQ ID NO:9, and a VL CDR3 comprising the amino acid sequence of SEQ ID NO:10.

2. A method of treating a solid tumor in a human subject, the method comprising administering to the subject a pharmaceutical composition comprising (i) antibodies or antigen-binding fragments thereof, wherein the antibodies or antigen-binding fragments thereof specifically bind to human B7-H4 and comprise a VH CDR1 comprising the amino acid sequence of SEQ ID NO:5, a VH CDR2 comprising the amino acid sequence of SEQ ID NO:6, a VH CDR3 comprising the amino acid sequence of SEQ ID NO:7, a VL CDR1 comprising the amino acid sequence of SEQ ID NO:8, a VL CDR2 comprising the amino acid sequence of SEQ ID NO:9, and a VL CDR3 comprising the amino acid sequence of SEQ ID NO:10 and (ii) a pharmaceutically acceptable excipient, wherein at least 95% of the antibodies or antigen-binding fragments thereof in the composition are afucosylated, and wherein about 0.005 to about 20 mg/kg of the antibodies or antigen-binding fragments thereof are administered.

3. The method of claim 1, wherein about 20 mg/kg of the antibody or antigen-binding fragment thereof is administered to the subject.

4-11. (canceled)

12. The method of claim 1, wherein the antibody or antigen-binding fragment thereof is administered about once every three weeks.

13. The method of claim 1, wherein the antibody or antigen-binding fragment thereof is administered intravenously.

14. The method of claim 1, wherein B7-H4 has been detected in the solid tumor using immunohistochemistry (IHC) prior to the administration.

15. The method of claim 1, wherein the antibody or antigen-binding fragment thereof comprises a VH comprising the amino acid sequence set forth in SEQ ID NO:11 and/or a VL comprising the amino acid sequence set forth in SEQ ID NO:12.

16. The method of claim 1, wherein the antibody or antigen-binding fragment comprises a heavy chain constant region and/or a light chain constant region.

17. (canceled)

18. The method of claim 1, wherein the antibody or antigen-binding fragment thereof comprises a heavy chain constant region comprising the amino acid sequence set forth in SEQ ID NO:25 and/or a light chain constant region comprising the amino acid sequence set forth in SEQ ID NO:23.

19. The method of claim 1, wherein the antibody or antigen-binding fragment thereof comprises a heavy chain comprising the amino acid sequence set forth in SEQ ID NO:21 and/or a light chain comprising the amino acid sequence set forth in SEQ ID NO:22.

20. (canceled)

21. The method of claim 1, wherein the antibody or antigen-binding fragment thereof is afucosylated.

22. The method of claim 1, wherein the antibody or antigen-binding fragment thereof is a full length antibody.

23. The method of claim 1, wherein the antibody or antigen-binding fragment thereof is an antigen binding fragment.

24-26. (canceled)

27. The method of claim 1, wherein the solid tumor is unresectable, locally advanced, and/or metastatic.

28. The method of claim 1, wherein the solid tumor is selected from the group consisting of breast cancer, ductal carcinoma, endometrial carcinoma, ovarian cancer, urothelial cancer, non-small cell lung cancer, pancreatic cancer, thyroid cancer, kidney cancer and bladder cancer.

29-30. (canceled)

31. The method of claim 28, wherein the breast cancer is a HER2-negative breast cancer, a triple negative breast cancer, a hormone-receptor (HR)-positive breast cancer, or a HER2-negative/HR-positive breast cancer.

32-34. (canceled)

35. The method of claim 1, wherein the subject has not received prior therapy with a PD-1/PD-L1 antagonist.

36. The method of claim 1, wherein the method further comprises monitoring the number of immune cells in the tumor.

37-39. (canceled)

40. A method of treating a solid tumor in a human subject, the method comprising intravenously administering to the subject about once every three weeks about 20 mg/kg of an antibody or antigen-binding fragment thereof that specifically binds to human B7-H4 and comprises a VH comprising the amino acid sequence set forth in SEQ ID NO:11 and a VL comprising the amino acid sequence set forth in SEQ ID NO:12.

41-42. (canceled)

43. The method of claim 40, wherein the solid tumor is breast cancer, ovarian cancer, endometrial cancer, or urothelial cancer.


SEQUENCE LISTING (31 sequences)

SEQ ID NO: 1 - protein, 282 aa, Artificial Sequence
  Human B7-H4
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 2 - protein, 282 aa, Artificial Sequence
  Cynomolgus monkey B7-H4
MASLGQILFWSIISIIFILAGAIALIIGFGISGRHSITVTTVASAGNIGEDGILSCTFEPDIKLSDIVIQWLKEGVIGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFLAISWALLPLAPYLMLK

SEQ ID NO: 3 - protein, 283 aa, Artificial Sequence
  Murine B7-H4
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFVAGWALLSLSCCLMLR

SEQ ID NO: 4 - protein, 282 aa, Artificial Sequence
  Rat B7-H4
MASLGQIIFWSIINVIIILAGAIVLIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIHTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLELLNSGPSPCVSSVSAAGWALLSLSCCLMLR

SEQ ID NO: 5 - protein, 11 aa, Artificial Sequence
  VH CDR1
GSIKSGSYYWG

SEQ ID NO: 6 - protein, 16 aa, Artificial Sequence
  VH CDR2
NIYYSGSTYYNPSLRS

SEQ ID NO: 7 - protein, 12 aa, Artificial Sequence
  VH CDR3
AREGSYPNQFDP

SEQ ID NO: 8 - protein, 11 aa, Artificial Sequence
  VL CDR1
RASQSVSSNLA

SEQ ID NO: 9 - protein, 7 aa, Artificial Sequence
  VL CDR2
GASTRAT

SEQ ID NO: 10 - protein, 9 aa, Artificial Sequence
  VL CDR3
QQYHSFPFT

SEQ ID NO: 11 - protein, 120 aa, Artificial Sequence
  VH 20502
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 12 - protein, 107 aa, Artificial Sequence
  VL 20502
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 13 - protein, 26 aa, Artificial Sequence
  VH FR1
QLQLQESGPGLVKPSETLSLTCTVSG

SEQ ID NO: 14 - protein, 14 aa, Artificial Sequence
  VH FR2
WIRQPPGKGLEWIG

SEQ ID NO: 15 - protein, 30 aa, Artificial Sequence
  VH FR3
RVTISVDTSKNQFSLKLSSVTAADTAVYYC

SEQ ID NO: 16 - protein, 11 aa, Artificial Sequence
  VH FR4
WGQGTLVTVSS

SEQ ID NO: 17 - protein, 23 aa, Artificial Sequence
  VL FR1
EIVMTQSPATLSVSPGERATLSC

SEQ ID NO: 18 - protein, 15 aa, Artificial Sequence
  VL FR2
WYQQKPGQAPRLLIY

SEQ ID NO: 19 - protein, 32 aa, Artificial Sequence
  VL FR3
GIPARFSGSGSGTEFTLTISSLQSEDFAVYYC

SEQ ID NO: 20 - protein, 10 aa, Artificial Sequence
  VL FR4
FGGGTKVEIK

SEQ ID NO: 21 - protein, 450 aa, Artificial Sequence
  Full-Length Heavy Chain Amino Acid Sequence 20502
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 214 aa, Artificial Sequence
  Full-Length Light Chain Amino Acid Sequence 20502
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 107 aa, Artificial Sequence [sequence omitted]

SEQ ID NO: 24 - DNA, 321 bp, Artificial Sequence
  constant region of a human kappa light chain nucleotide
cggaccgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - protein, 330 aa, Artificial Sequence [sequence omitted]

SEQ ID NO: 26 - DNA, 990 bp, Artificial Sequence
  constant region of a human IgG1 heavy chain nucleotide sequence
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 27 - DNA, 360 bp, Artificial Sequence
  Heavy Chain Variable Region-Encoding Polynucleotide Sequence 20502
cagctgcagctgcaggagtcgggcccaggactggtgaagccttcggagaccctgtccctcacctgcactgtctctggtggctccatcaaaagtggtagttactactggggctggatccgccagcccccagggaaggggctggagtggattgggaacatctattatagtgggagcacctactacaacccgtccctcagaagtcgagtcaccatatccgtagacacgtccaagaaccagttctccctgaagctgagttctgtgaccgccgcagacacggcggtgtactactgcgccagagaaggatcttaccccaatcagtttgatccatggggacagggtacattggtcaccgtctcctca

SEQ ID NO: 28 - DNA, 321 bp, Artificial Sequence
  Light Chain Variable Region-Encoding Polynucleotide Sequence 20502
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtaccactccttccctttcacttttggcggagggaccaaggttgagatcaaa

SEQ ID NO: 29 - protein, 385 aa, Artificial Sequence
  B7-H4 IgV-huIgG1
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 288 aa, Artificial Sequence
  hPD-1 sequence
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 31 - protein, 290 aa, Artificial Sequence
  hPD-L1
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET